(19) United States
(12) Patent Application Publication
(10) Pub. No.: US 2022/0395160 A1
(43) Pub. Date: Dec. 15, 2022

(54) HANDLE FOR AN ENDOSCOPE

(71) Applicant: 270 SURGICAL LTD., Netanya (IL)

(72) Inventors: Golan SALMAN, Atlit (IL); Itay SIDAR, Haifa (IL); Yuri GERSHOV, Zikhron Yaakov (IL); Hadar SCHWARCZ, Netanya (IL); Leonid KRIVOPISK, Nesher (IL)

(21) Appl. No.: 17/642,014

(22) PCT Filed: Sep. 8, 2020
(86) PCT No.: PCT/IL2020/050976

§ 371 Date: Mar. 10, 2022

Related U.S. Application Data
(60) Provisional application No. 62/898,619, filed on Sep. 11, 2019.
(60) Provisional application No. 62/935,429, filed on Nov. 14, 2019.

(51) Int. Cl. A61B 1/00; A61B 1/06; A61B 1/04

(52) U.S. Cl. CPC A61B 1/00066; A61B 1/00006; A61B 1/0684; A61B 1/042

(57) ABSTRACT

Provided herein is a handle of a multi-camera endoscope, the handle includes a user interface activating unit which includes one or more external activating/control buttons, and an internal activating element, the handle optionally includes a converter configured to receive one or more parallel signals and transform the one or more parallel signals to one or more corresponding serial signals, wherein the parallel signals are output by one or more cameras of the endoscope. Further provided are endoscopes and medical imaging system which include the handle, as well as methods of use thereof.

### TECHNICAL FIELD

The present disclosure relates generally to handle of an endoscope, in particular a handle having magnetic activating buttons, the handle is optionally configured to process, convert and relay digital signals obtained from the distal tip of the endoscope, within the handle.

### BACKGROUND

An endoscope is a medical device used to image an anatomical site (e.g. a body cavity, a hollow organ). Unlike some other medical imaging devices, the endoscope is inserted into the anatomical site (e.g. through small incisions made on the skin of the patient). An endoscope can be employed not only to inspect an anatomical site and organs therein (and diagnose a medical condition in the anatomical site) but also as a visual aid in surgical procedures. Medical procedures involving endoscopy include laparoscopy, arthroscopy, cystoscopy, ureteroscopy, and hysterectomy.

There is a need in the art for an improved handle of an endoscope which can allow improved control of the operation of the endoscope by utilizing sensitive and versatile user interface activating unit allowing a user to operate the endoscope via the handle in an efficient and sensitive manner, and optionally further improve data processing, obtained from multi lens assemblies and sensors (e.g. cameras) of the endoscope.

### SUMMARY

Aspects of the disclosure, according to some embodiments thereof, relate to a handle of an endoscope, wherein the handle is configured to control operation of the endoscope and to further process signals received from the endoscope distal tip, wherein the signals are processed within the handle, prior to the signals being transmitted to an external control unit.

According to some embodiments thereof, the handle of the endoscope is configured to efficiently and accurately allow a user to control operation of the endoscope utilizing a user interface activating unit which includes external magnetic buttons, allowing interaction and control of cameras of the endoscope on one end and/or interaction with an external control unit on the other end.

According to some embodiments, the user interface activating unit of a handle of an endoscope as disclosed herein is advantageous as it allows the accurate, convenient and sensitive operation of the endoscope, in particular of cameras located at the distal tip thereof, while maintaining the integrity, sterility and sealing of the handle. In some embodiments, the handle further allows the advantageous processing of signals received from the cameras at the distal tip of the endoscope, and further conveying/relaying those signals to an external control unit, to further process the signals, to result in enhanced image processing and displaying of the obtained data by the external control unit.

According to some embodiments, the handle of an endoscope disclosed herein is advantageous as it allows not only the accurate, convenient and sensitive operation of the endoscope, but also the advantageous processing of signals (for example, parallel signals) received from cameras at the distal tip of the endoscope to serial signals, and further conveying/relaying those signals to an external control unit, to further process the signals, to result in enhanced image processing and displaying of the obtained data by the external control unit.

In some embodiments, for transferring data, such as images, from the camera to a control unit, the serial data method may be preferred, compared to parallel as-is data, which is more problematic. Thus, if using sensors which provide parallel data, it is advantageous to convert said parallel data to serial data, within the handle, for a more robust transferring of information to the control unit. In some embodiments, serialization may be advantageous for various reasons. For example, serialization can minimize cable count/number which results in: better EMC performance (also due to higher bit rate); better cross-talk immunity; simpler, cheaper, lighter, more flexible, thinner cable; smaller and more cost effective connectors; Simpler Host logic and pin count (for example, only three RX transceivers needed may be needed, instead of dozens of general-purpose input/output GPIOs); simpler host analog interfaces (for example, VGA/EQ/CDR), instead of dozens of GPIOs. Additional advantages of serialization may be attributed to clock related parameters of the serialized data, embedded in the data, for example, dedicated cable for clock is redundant; clock-data skew issues are avoided, and the like.

In some embodiments, the handle disclosed herein exhibits an advantageous architectural internal structuring, that allows the accommodation of the various electrical and mechanical components required for the operation of the handle, within the limited internal space of the handle, while achieving an efficient mode of action (for example, in signal processing and communication of the signals) and advantageously, allow a very accurate and sensitive control, resulting in improved control/operation/activation of the endoscope by the user. The internal architectural structuring of the handle and in particular the user interface activating unit advantageously allow maintaining a sealed environment within the handle (i.e. preventing fluids from entering the internal regions of the handle), and furthermore allow a more sensitive and accurate control of operation by a user generating excessive amount of internal heat.

According to some embodiments, the handle disclosed herein includes one or more Printed Circuit Boards, also known as PCBs, (for example, three PCBs), that may be placed in any suitable orientation, for example, parallel to each other (along the longitudinal axis of the handle), the PCBs may be interconnected to each other (functionally and or physically), as well as to other components (such as, cameras and/or signal convertors/transmitters), to ultimately allow the activity of the handle, both in controlling the endoscope operation and in signal processing, as further detailed herein.

According to some embodiments, there is provided a handle of a multi-camera endoscope, the handle includes a conversion unit/converter configured to receive one or more parallel signals and transform said one or more parallel signals to one or more corresponding serial signals, wherein the parallel signals are output by one or more cameras of the endoscope, said conversion unit/converter is further configured to transmit or relay the serial signals to an external control unit.

According to some embodiments, there is provided a handle of a multi-camera endoscope, the handle includes a user interface activating unit configured to control one or more operating parameters of the endoscope, the user interface activating unit, also referred to as an activation unit includes external operation/control buttons, configured to interact with a corresponding internal activation element, said activation having corresponding sensors configured to interact with the external operation buttons to convey operating commands to one or more cameras of the endoscope, to thereby control one or more operation parameters of the endoscope.

According to some embodiments, there is provided a handle of a multi-camera endoscope, the handle includes a user interface activating unit configured to control one or more operating parameters of the endoscope, the user interface activating unit includes one or more external activating/control buttons having a magnetic region, and an internal activating element, said internal activating element includes one or more magnetic sensors, each of said one or more magnetic sensors is configured to interact with a corresponding external control button, to convey operating commands to the endoscope, to thereby facilitate control of one or more operation parameters of the endoscope.

According to some embodiments, the one or more magnetic sensors are selected from an analog magnetic sensor and a linear magnetic sensor.

According to some embodiments, the analog magnetic sensor may be located at a distance of about 0.5 to about 0.7 millimeters from the magnetic region of the corresponding external activating/control button at a non-activated state (off state).

According to some embodiments, the analog magnetic sensor may be activated when the distance between the analog sensor and the magnetic region of the corresponding external control button is changed by about 0.6 millimeters

According to some embodiments, the linear magnetic sensor may be activated when a change is voltage activation of the linear magnetic sensor is sensed.

According to some embodiments, the change in voltage may be in the range of about 0.2-0.5V.

According to some embodiments, the external control button and the corresponding internal activating element do not physically interact.

According to some embodiments, the handle includes at least two external control buttons and at least two corresponding internal activating buttons.

According to some embodiments, the internal activating element may be mounted on a Printed Circuit Board (PCB), located within the handle.

According to some embodiments, the handle may further include a converter configured to receive one or more parallel signals and transform said one or more parallel signals to one or more corresponding serial signals, wherein the parallel signals are output by one or more optical sensors of the endoscope, said converter is further configured to transmit or relay the serial signals to an external control unit.

According to some embodiments, the converter may be configured to relay the serial signals to a utility cable connected to the external control unit that is functionally associated with the handle.

According to some embodiments, the one or more parallel signals received at the converter may be at 8-12 bit, at 9-11 bit, and/or at 10 bit.

According to some embodiments, the one or more corresponding serial signals may be transmitted at a rate of about 1-2 Giga bits per second, or a rate of about 1.5 Giga bits per second.

According to some embodiments, the external control unit may be connected to a proximal end of the handle.

According to some embodiments, the one or more optical sensors are located at a distal section of an elongated shaft of the endoscope and each of the one or more optical sensors is associated with a lens assembly located at distal section to obtain a camera, the elongated shaft being mounted on the distal end of the handle.

According to some embodiments, the transmitting to the control unit is direct or via one or more intermediate elements.

According to some embodiments, the converter is or includes a Field Programmable Gate Array (FPGA).

According to some embodiments, the handle may include or more Printed Circuit board(s) (PCBs), communicatively associated with the sensors.

According to some embodiments, the handle includes at least two PCBs.

According to some embodiments, the PCBs may include rigid PCB, flexible PCB, or both.

According to some embodiments, the at least two PCB's are placed in parallel along a longitudinal axis of the handle.

According to some embodiments, the handle may include at least three PCBs.

According to some embodiments, the converter may be mounted on a PCB.

According to some embodiments, each of the one or more optical sensors may be connected to transferring means, extending along the elongated shaft of the endoscope and connected to the converter. According to some embodiments, the transferring means may be selected from, but not limited to: cables, flexible PCBs, rigid PCBs, flex-rigid PCBs or any combination therefore.

According to some embodiments, the handle further may further include one or more secondary power sources, configured to receive power from a primary external power source and distribute power within the handle.

According to some embodiments, the handle may further include one or more of: a micro-controller (controller), a universal asynchronous receiver-transmitter (UART), a mounting element, a connecting element, or any combination thereof.

According to some embodiments, the handle may be functionally associated with an external control unit, said external control unit is connected to a proximal end of the handle.

According to some embodiments, the endoscope includes one or more optical sensors located at a distal section of a shaft of the endoscope and wherein each of the one or more optical sensors is associated with a lens assembly to obtain a camera, the shaft being mounted on the distal end of the handle.

According to some embodiments, the endoscope includes one or more illumination components located at the distal section of the elongated shaft of the endoscope.

According to some embodiments, the control of the operating parameters includes controlling the operation of the one or more optical sensors, one or more lens assemblies, cameras, illumination component(s), or any combination thereof.

According to some embodiments, the operating parameters may include: zoom-in, zoom out, image-capture, freezing an image, recording an image or a video, saving an image of a videos, changing the intensity of the illumination component, manipulating between video streams of the multi-camera endoscope to be displayed on a monitor, or any combination thereof.

According to some embodiments, the handle is essentially sealed, such that external fluids are prevented from entering internal space of the handle. According to some embodiments, the handle may further include a leak tester.

According to some embodiments, there is provided an endoscope having the handle as disclosed herein, and an elongated compatible shaft.

According to some embodiments, the endoscope includes at least one camera and at least one illumination component associated with said at least one camera located at the elongated shaft distal section. According to some embodiments, the endoscope includes at least two cameras.

According to some embodiments, each of the cameras is connected to transferring means, extending along the elongated shaft of the endoscope and connected to the converter located in the handle.

According to some embodiments, the at least two cameras include a front camera on a distal tip of the elongated shaft and a first side-camera. According to some embodiments, the at least two cameras further includes a second side-camera, wherein the first side-camera and the second side-camera are positioned on opposite sides of the elongated shaft, and wherein the first side-camera is positioned closely relative to the second side-camera.

According to some embodiments, the at least two cameras provide at least about 270 degrees horizontal field-of-view (FOV) of a target area within an anatomical cavity into which the elongated shaft is inserted.

According to some embodiments, the at least one illumination component is or comprises a discrete light source.

According to some embodiments, the elongated shaft is rigid or semi rigid.

According to some embodiments, there is provided a method for controlling operation of a medical imaging system including a multi-camera endoscope, the method includes conveying operation instructions from user interface activating unit, located within a handle of the multi-camera endoscope, wherein the operation instructions are further conveyed to a main control unit, and/or to one or more cameras and/or to one or more illumination components located at a distal end of the endoscope.

According to some embodiments, the operating parameters include one or more of: zoom in, zoom out, image capture, freezing an image, saving an image, recording a video, changing the intensity of the light source, manipulating between video streams of the multi-camera endoscope to be displayed on a monitor, or any combination thereof.

According to some embodiments, there is provided a method for controlling operation of a multi camera of an endoscope, the method includes conveying operation instructions from one or more external activating/control buttons located on an external surface of a handle of the multi camera endoscope to one or more corresponding sensors of an internal activating element, located within the handle, wherein the external control button and the corresponding activating element to do not physically interact, wherein the operation instructions are further conveyed to one or more cameras and/or one or more illumination components, located at a distal end of an elongated shaft of the endoscope. According to some embodiments, the one or more external control buttons include a magnetic region. According to some embodiments, the one or more sensors includes magnetic sensors. According to some embodiments, the magnetic sensors may be selected from analog magnetic sensor and linear magnetic sensor. According to some embodiments, the operating parameters includes one or more of: zoom in, zoom out, image capture, freezing an image, saving an image, recording a video, changing the intensity of the light source, manipulating between video streams of the multi-camera endoscope to be displayed on a monitor, or any combination thereof.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1A shows a schematic, perspective side view of a handle, according to some embodiments;

FIG. 1B shows a schematic, perspective view of a rigid endoscope including a handle, and an elongated shaft, according to some embodiments;

FIG. 2 schematically depicts a medical imaging system including an endoscope and a handle, according to some embodiments;

FIG. 3 schematically depicts an elongated shaft of an endoscope, and a field-of-view provided by cameras positioned in a distal section of the elongated shaft, according to some embodiments;

FIGS. 4A and 4B show block diagrams of a handle, interacting with a main control unit and a distal tip of a medical imaging system, according to FIG. 2;

FIG. 5A is a schematic perspective cross sectional view of an endoscope handle, according to some embodiments;

FIGS. 5B-5C are schematic perspective cross sectional views of an endoscope handle, according to FIG. 5A; and

FIG. 6 is a schematic, perspective view of internal setting (architecture) of three PCB elements of an endoscope handle, according to some embodiments;

FIGS. 7A-B show schemes of operation of an analog magnetic sensor (FIG. 7A) and operation of a linear magnetic sensor (FIG. 7B), according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

According to some embodiments, there is provided herein an advantageous handle of a multi-camera endoscope, the handle includes a user interface activating unit which includes an external interface (in the form of activation/control/operation buttons) and an internal interface in the form of internal activating unit having suitable sensors configured to interact with the external control buttons, to allow a user to control the endoscope in a safe, accurate and sensitive manner.

According to some embodiments, there is provided herein an advantageous handle of a multi-camera endoscope, the handle includes a conversion/processing unit configured to receive one or more parallel signals which are output by one or more (for example, at least two) cameras of the endoscope and to transform/convert the one or more parallel signals to one or more corresponding serial signals, within the handle, wherein the conversion unit/converter may be further configured to transmit or relay the serial signals generated in the handle to an external control unit, for further processing (such as, image manipulation, image display, etc.).

According to some embodiments, there is provided herein an advantageous handle of a multi-camera endoscope, the handle includes a user interface activating unit which includes an external interface (in the form of activation/control/operation buttons) and an internal interface in the form of internal activating unit having suitable sensors configured to interact with the external control buttons wherein the handle may optionally further include a conversion/processing unit configured to receive one or more parallel signals which are output by one or more (for example, at least two) cameras of the endoscope and to transform/convert the one or more parallel signals to one or more corresponding serial signals, within the handle, wherein the conversion unit/converter may be further configured to transmit or relay the serial signals generated in the handle to an external control unit, for further processing (such as, image manipulation, image display, etc.).

As used herein, the term “PCB” is a printed circuit board. PCB mechanically supports and electrically connects electronic or electric components using conductive tracks, pads and the like, etched from one or more sheet layers of copper laminated onto and/or between sheet layers of a non-conductive substrate. Components may be soldered onto the PCB to both electrically connect and mechanically fasten them to it. In some embodiments, the term PCB also includes a PCB assembly, i.e. a printed circuit board having various components (such as, electrical components) placed/associated with/mounted thereon. In some exemplary embodiments, the PCB may be rigid and/or semi rigid. In some embodiments, the PCB may be flexible and/or semi flexible. In some embodiments, the PCBs may be mounted in specific orientation within the handle. In some embodiments, the PCB maybe mounted in parallel (or essentially parallel) to the longitudinal axis of the handle. In some embodiments, the PCB may be mounted vertically (or essentially vertically) to the longitudinal axis of the handle. In some embodiments, more than one PCB may be mounted in the handle. In some embodiments, when more than one PCBs is mounted in the handle, at least some of the PCBs may be placed in parallel and some vertically with respect to the longitudinal axis of the handle.

As used herein, the term “UART” relates to a universal asynchronous receiver-transmitter.

As used herein, the term “FPGA” refers to Field-Programmable Gate Array. In some embodiments, the FPGA is a converter, configured to convert parallel signal to serial signal, and optionally further relay (directly or indirectly) the generated serial signal. In some exemplary embodiments, the FPGA is positioned/mounted/placed on a PCB.

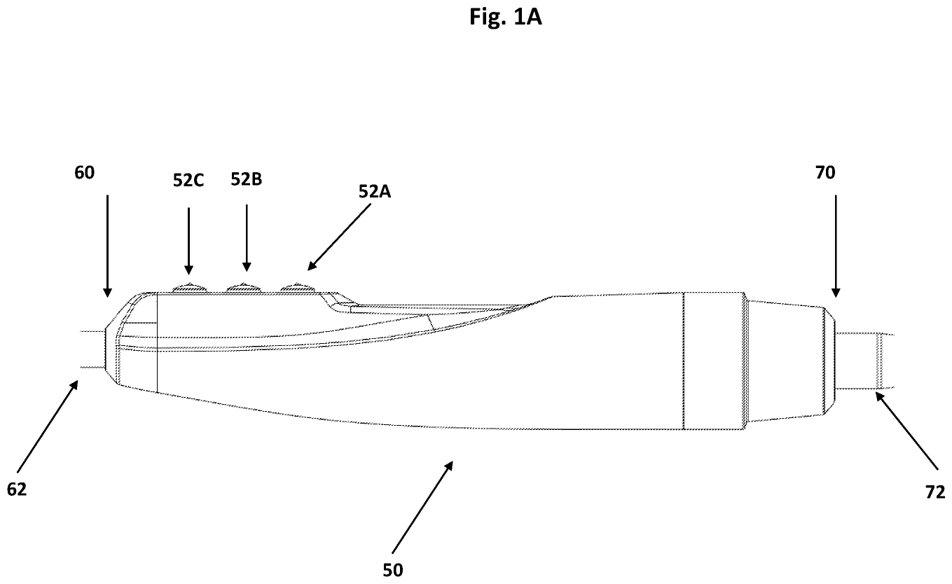

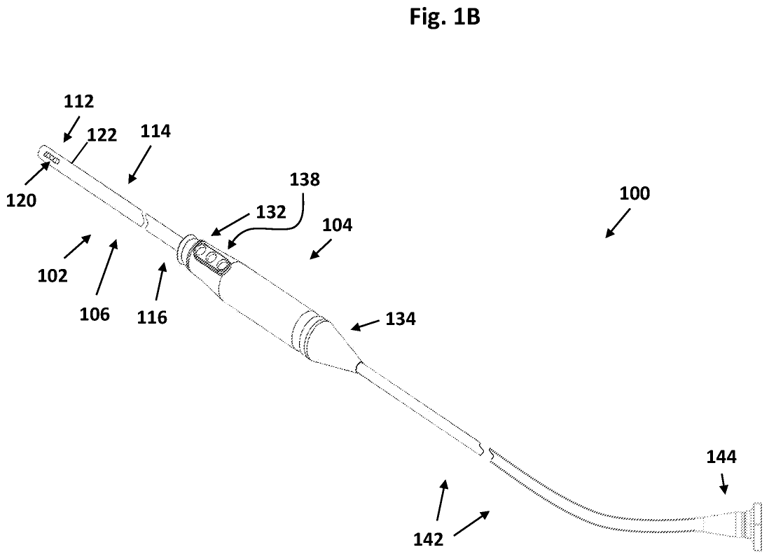

Reference is now made to FIG. 1A, which schematically shows a perspective view of a handle, according to some embodiments. As shown in FIG. 1A, handle, 50 includes a distal end, 60 and a proximal end, 70. To the distal end of the handle, a shaft (the proximal end of shaft 62 is shown in FIG. 1A) is connected. The shaft is configured to be inserted into a body cavity, as detailed below. At its proximal end, the handle may be connected to external main control unit, via, for example, connecting interface (that may include, adaptors, connectors, cables, wires, and the like). A distal end, 72, of such connecting cable (also referred herein as utility cable) is shown in FIG. 1A. The handle may further include a suitable outer casing that may be advantageously sealed to fluids, such as, liquids and gases. The handle further includes a user interface activating unit, which includes external exemplary activating/operating/control buttons 52A-C, configured to allow operating the handle and hence the endoscope, as further detailed below. In some embodiments, the handle may be configured to (i) afford a user (e.g. a surgeon) a comfortable and secure grip of the handle, and/or (ii) to be manipulated by a robotic arm or robotic gripping means (e.g. controlled by the surgeon).

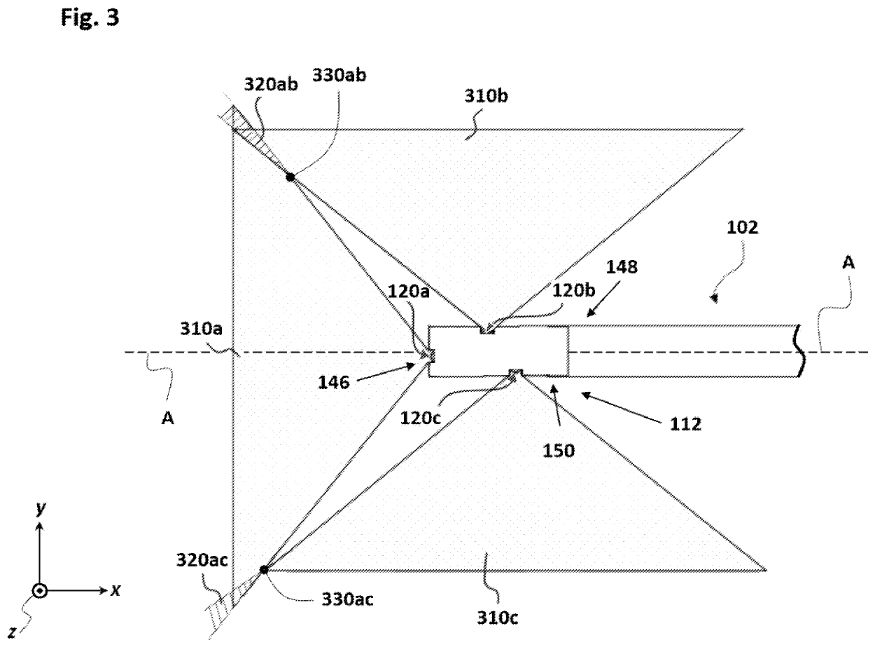

Reference is now made to FIG. 1B, which schematically depicts a rigid endoscope, according to some embodiments. As shown in FIG. 1B, endoscope 100 includes an elongated shaft 102, configured to be inserted into an anatomical site (e.g. an anatomical cavity), and a handle 104, configured to be held by a user (e.g. a surgeon) of endoscope 100 and to facilitate guiding and manipulation of elongated shaft 102 (particularly a distal section thereof) within the anatomical site. As detailed herein, handle 104 may be further configured to convert parallel signals to serial signal and relay them to a control unit. Elongated shaft 102 is configured to be integrated within handle 104. In some embodiments, elongated shaft 104 may be detachably mountable on handle 104. Shaft 102 includes a shaft body 106, e.g. a rigid tubular member. Shaft 102 includes a shaft distal section 112, a shaft central section 114, and a shaft proximal section 116 (i.e. a distal section, a central section, and a proximal section, respectively, of shaft 102). Shaft distal section 112 includes at least two cameras 120 (e.g. a front camera, as seen for example in FIG. 3, and at least one side camera) and illumination components 122, such as light emitting diodes (LEDs).

According to some embodiments, each of illumination components 122 is or includes a discrete light source. According to some embodiments, illumination components 122 may be mounted on one or more PCBs in shaft distal section 112. According to some embodiments, wherein illumination components 122 include LEDs, the LEDs may include, for example, one or more white light LEDs, infrared LEDs, a near infrared LEDs, an ultraviolet LED, and/or a combination thereof. It is noted that in embodiments wherein illumination components include LEDs configured to produce light outside the visible spectrum (e.g. an infrared spectrum, a UV spectrum), cameras 120 will include sensors configured to detect such light (e.g. infrared light, ultraviolet). That is, cameras 120 will have capacities of e.g. infrared cameras and so on.

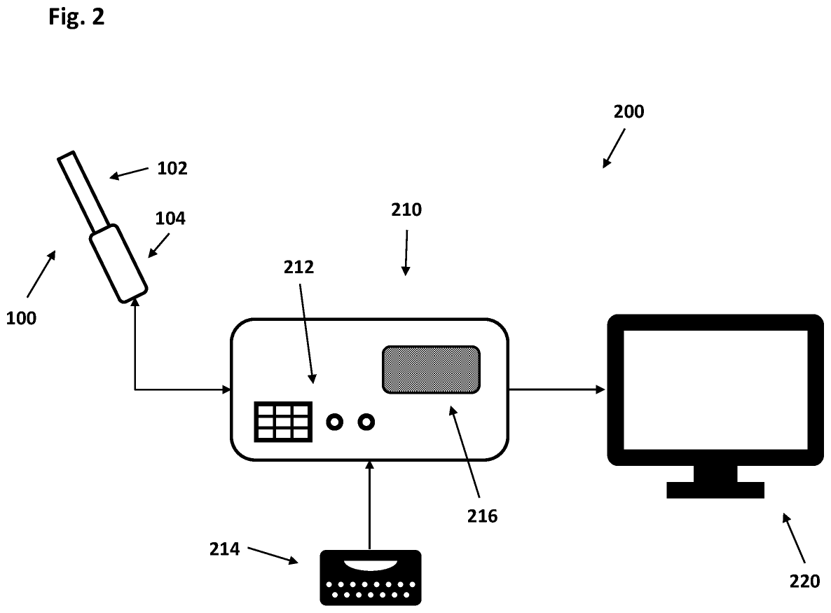

According to some embodiments, the illumination components may include the distal tips of respective optical fibers (not shown). According to some such embodiments, handle 104 may include one or more light sources connected to one or more optical fibers extending through handle 104 and shaft 102. The optical fibers are configured to guide the light produced by the light sources from handle 104 to shaft distal section 112, wherefrom the guided light may be shone such as to illuminate the field-of-view of cameras 120. According to some embodiments, the light sources may be external to handle 104, being positioned, for example, in a main control unit such as the main control unit depicted in FIG. 2. Handle 104 includes a handle distal section 132 and a handle proximal section 134 (i.e. a distal section and a proximal section of handle 104, respectively).

Handle distal section 132 may include a user control interface 138 (such as user interface activating unit, which includes external exemplary activating/operating/control buttons 52 and internal interface in the form of internal activating unit (not shown) according to FIG. 1A) configured to allow a user to control endoscope 100 functions. User control interface 138 may be functionally associated with cameras 120 and illumination components 122 via an electronic coupling between shaft 102 and handle 104. According to some embodiments, user control interface 138 may allow, for example, to control zoom, focus, record/stop recording, and/or freeze frame functions of cameras 120 and/or to adjust the light intensity provided by illumination components 122. According to some embodiments, user control interface 138 may allow to control the presentation of video streams from cameras 120 on an associated monitor (such as the monitor depicted in FIG. 2). For example, according to some embodiments, user control interface 138 may allow to present the video streams side-by-side, and/or with a video stream from one of the cameras (e.g., a front camera, at least one side camera) being displayed larger than the video streams from the other cameras (e.g., front camera, at least one side camera), and/or to display two copies of one of the video streams and allow to manipulate one of the copies. User control interface 138 may include one or more buttons 140 (as a non-limiting example, three buttons, as depicted in FIGS. 1A and 1B; not all of which are numbered), knobs, switches, a touch panel, and/or the like.

Each of cameras 120 may include a sensor, such as a charge coupled device (CCD) image sensor or a complementary metal oxide semiconductor (CMOS) image sensor, and a camera lens (e.g. an extreme wide-angle lens) or a lens assembly. According to some embodiments, each of the sensors may be mounted on a respective printed circuit board (PCB). According to some embodiments, all the sensors may be mounted on a common PCB. In some embodiments, the sensors may be located within handle 104. Cameras 120 may be configured to provide a continuous/panoramic/surround field-of-view (FOV), as elaborated on below in the description of FIG. 3.

Reference is now made to FIG. 2, which schematically depicts a medical imaging system 200, according to some embodiments. Medical imaging system 200 includes a multi camera endoscope 100, having, for example at least two cameras, a main control unit 210, and a monitor 220. According to the convention adopted herein, a same reference numeral in FIGS. 1B, 2 and 3, refers to the same object (e.g. device, element). Thus, for example, in FIGS. 1, and 2, the reference numeral 100 refers to the same endoscope (i.e. endoscope 100). Similarly, in FIGS. 1B, 2 and 3, the reference numeral 102 refers to the same shaft (i.e. shaft 102 of endoscope 100).

Endoscope 100 and monitor 220 may each be functionally associated with main control unit 210. Main control unit 210 includes processing circuitry (e.g. one or more processors and memory components) configured to process (digital data) from cameras 120 (not shown in FIG. 2 but depicted in FIG. 1), such as to display the captured images and video streams on monitor 220. In particular, the processing circuitry may be configured to process the digital data received from each of cameras 120, such as to produce therefrom video files/streams providing a panoramic/surround view of the anatomical site, as explained below in the description of FIG. 3. According to some embodiments, the processing circuitry may be configured to process the data received from cameras 120 to produce a combined video stream providing a continuous and consistent (seamless) panoramic view of the anatomical site.

Main control unit 210 may include a user interface 212 (e.g. buttons and/or knobs, a touch panel, a touch screen) configured to allow a user to operate main control unit 210 and/or may allow control thereof using one or more input devices 214, e.g. an external user control interface connectable thereto such as a keyboard, a mouse, a portable computer, and/or even a mobile computational device e.g. a smartphone or a tablet. According to some embodiments, input devices 214 may include a voice controller. According to some embodiments, main control unit 210 may further be configured to partially or even fully operate cameras 120 and illumination components 122 (shown in FIG. 1i). Some operational aspects may be operated automatically, for example, according to some embodiments, the supply of power to endoscope 100 components, such as cameras 120 and illumination components 122, while other operational aspects or functions may be operated using user interface 212 and/or input devices 214. According to some embodiments, main control unit 210 may include a display 216 (for example, the touch screen and/or another screen) for presenting information regarding the operation of endoscope 100, such as the brightness levels of cameras 120, zoom options, focus, and the like. According to some embodiments, wherein display 216 is a touch screen, display 216 may further allow controlling for example, the zoom, focus, record/stop recording functions, freeze frame function, and/or the brightness of cameras 120, and/or to adjust the light intensity of illumination components 122. According to some embodiments, the choice of information presented may be controlled using user interface 212, user control interface 138, and/or input devices 214.

According to some embodiments, endoscope 100 is functionally associated with main control unit 210 via a utility cable 142 (shown in FIGS. 1A and 1B) connected to or configured to be connected to handle proximal section 134, and further configured to be connected to main control unit 210 (via, for example, a plug 144 or a port). Utility cable 142 may include at least one data cable for receiving video signals from cameras 120, and at least one power cable for providing electrical power to cameras 120 and to illumination components 122, as well as to operationally control parameters of cameras 120 and illumination components 122, such as the light intensity. Additionally or alternatively, according to some embodiments, endoscope 100 may include a wireless communication unit (e.g. a Bluetooth antenna or Wi-Fi) configured to communicatively associate endoscope 100 with main control unit 210. According to some embodiments, endoscope 100 is configured to be powered by a replaceable and/or rechargeable battery included therein, i.e. inside handle 104. According to some embodiments, wherein illumination components 122 include the distal tips of optical fibers and wherein the light source(s) is positioned in main control unit 210, cable 142 will also include one or more optical fibers configured to guide the light produced by the light source(s) to an optical fiber(s) in handle 104, wherefrom the light will be guided to optical fibers in shaft 102.

Monitor 220 is configured to display images and, in particular, to display stream videos captured by cameras 120, and may be connected to main control unit 210 by a cable (e.g. a video cable) or wirelessly. According to some embodiments, monitor 220 may be configured to display thereon information regarding the operation of endoscope 100, as specified above. According to some embodiments, monitor 220, or a part thereof, may function as a touch screen. According to some such embodiments, the touch screen may be used to operate main control unit 210. According to some embodiments, images/videos from different cameras (from cameras 120) may be displayed separately (e.g. side-by-side, picture on picture, in an equal aspect ratio, in an un-equal aspect ratios, in multiple copies of one or more of the video streams, and the like) on monitor 220, and/or may be presented as a single panoramic image/video. According to some embodiments, user interface 212 and/or input devices 214 and/or user control interface 138 are configured to allow switching between images/videos corresponding to different FOVs (of different cameras). For example, according to some embodiments, wherein cameras 120 include a front camera 120a, a first side-camera 120b, and a second side-camera 120c: switching between footage captured by front camera 120a to footage captured by first side camera 120b, switching between footage captured by front camera 120a to footage captured by second side-camera 120c, or switching between a panoramic video generated from the footage of all of cameras 120a, 120b, and 120c to footage captured by one of cameras 120a, 120b, or 120c. Cameras 120a, 120b, and 120c are depicted together in FIG. 3. According to some embodiments, main control unit 210 may be associated with a plurality of monitors, such as monitor 220, thereby allowing displaying different videos and images on each. For example, main control unit 210 may be associated with four monitors, such as to allow displaying videos from each of cameras 120a, 120b, 120c on three of the monitors, respectively, and a panoramic video (corresponding to the combination of the three videos) on the fourth monitor, which may be wider than the other three.

The field-of-view (FOV) provided by endoscope 100 is the combination of the respective FOVs provided by each of cameras 120. Cameras 120 may be configured to provide a continuous and consistent FOV, or at least a continuous and consistent horizontal FOV (HFOV), as explained below.

Reference is now made to FIG. 3, which schematically depicts shaft distal section 112 (of shaft 102) and a combined HFOV provided by front camera 120a, first side-camera 120b, and second side-camera 120c, according to some embodiments. Front camera 120a is positioned within shaft distal section 112 on a front surface 146 of the distal tip (not numbered) of shaft distal section 112, with a lens assembly (not numbered) of front camera 120a being exposed on front surface 146. First side-camera 120b is positioned within shaft distal section 112 on a first side-surface 148 thereof, with a lens assembly (not numbered) of first side-camera 120b being exposed on first side-surface 148. Second side-camera 120c is positioned within shaft distal section 112 on a second side-surface 150 thereof, with a lens assembly (not numbered) of second side-camera 120c being exposed on second side-surface 150. First side-surface 148 may be positioned opposite to second side-surface 150, essentially 180 degrees apart relative to shaft distal section 112 longitudinal axis A. According to some embodiments, first side-camera 120b and second side-camera 120c are not positioned back-to-back. According to some embodiments, the distance between the center-point of first side-camera 120b (i.e. the center of a lens assembly of first side-camera 120b) and front surface 146 is between about 5 millimeters to about 20 millimeters and the distance between the center-point of first side-camera 120b and the center-point of second side-camera 120c may be up to about 10 millimeters.

In some embodiments, the combined HFOV is formed by a front HFOV 310a, a first side-HFOV 310b, and a second side-HFOV 310c of front camera 120a, first side-camera 120b, and second side-camera 120c, respectively. Each of HFOVs 310a, 310b, and 310c lies on the xy-plane. HFOV 310a is positioned between HFOVs 310b and 310c and overlaps with each. A first overlap area 320ab corresponds to an area whereon HFOVs 310a and 310b overlap. In other words, first overlap area 320ab is defined by the intersection of the xy-plane with the overlap region (volume) of the FOVs of front camera 120a and first side-camera 120b. Similarly, a second overlap area 320ac corresponds to an area whereon HFOVs 310a and 310c overlap. A first intersection point 330ab is defined as the point in first overlap area 320ab which is closest to front camera 120a. It is noted that first intersection point 330ab also corresponds to the point in first overlap area 320ab which is closest to first side-camera 120b. Similarly, a second intersection point 330ac is defined as the point in second overlap area 320ac which is closest to front camera 120a. It is noted that second intersection point 330ac also corresponds to the point in second overlap area 320ac which is closest to second side-camera 120c.

The combined FOV (of cameras 120a, 120b, and 120c) is continuous since the panoramic view provided thereby does not contain any gaps (as would have been the case had HFOV 310a not overlapped with at least one of HFOVs 310b and 310c). Further, the combined HFOV is consistent (i.e. seamless) in the sense that the magnifications of the lenses of each of cameras 120a, 120b, and 120c are compatible such that the view of objects (e.g. organs or surgical tools), or parts of objects, in the overlap areas are not distorted and the (overall) combined HFOV merges the combined HFOVs of each front HFOV 310a and first side-HFOV 310b, and front HFOV 310a and second side-HFOV 310c, in a seamless manner. Thus, the magnification provided by the lens of first side-camera 120b may be slightly larger than the magnification provided by the lens of front camera 120a to compensate for first intersection point 330ab being closer to front camera 120a than to first side-camera 120b. That is, D₁<D₂, wherein D₁ is the distance between front camera 120a and first intersection point 330ab, and D₂ is the distance between first intersection point 330ab and first side-camera 120b. Similarly, the magnification provided by the lens of second side-camera 120c may be slightly larger than the magnification provided by the lens of front camera 120a to compensate for second intersection point 330ac being closer to front camera 120a than to second side-camera 120c.

According to some embodiments, the combined HFOV spans between about 220 degrees to about 270 degrees, between about 240 degrees to about 300 degrees, or between about 240 degrees to about 340 degrees. Each possibility corresponds to separate embodiments. According to some embodiments, the combined HFOV spans at least about 270 degrees. According to some embodiments, for example, each of HFOVs 310a, 310b, and 310c may measure between about 85 degrees to about 120 degrees, between about 90 degrees to about 110 degrees, or between about 95 degrees to about 120 degrees. Each possibility corresponds to separate embodiments.

According to some embodiments, shaft 102 may measure between about 100 millimeters and about 500 millimeters in length, and shaft body 106 may have a diameter measuring between about 2.5 millimeters and about 15 millimeters. According to some embodiments, front camera 120a may be offset relative to the longitudinal axis A, which centrally extends along the length of shaft 102. According to some embodiments, the distance between second side-camera 120c and front surface 146 is greater than the distance between first side-camera 120b and front surface 146.

According to some embodiments, front camera 120a may be offset relative to the longitudinal axis A by up to about 0.05 millimeters, up to about 0.1 millimeters, up to about 0.5 millimeters, up to about 1.0 millimeters, up to about 1.5 millimeters, up to about 5.0 millimeters, or up to about 7.0 millimeters. Each possibility corresponds to separate embodiments. According to some embodiments, for example, front camera 120a may be offset relative to the longitudinal axis A by between about 0.05 millimeters to about 0.1 millimeters, about 0.5 millimeters to about 1.5 millimeters, about 1.0 millimeter to about 5.0 millimeters, about 1.5 millimeters to about 5.0 millimeters, or about 1.0 millimeters to about 7.0 millimeters. Each possibility corresponds to separate embodiments. According to some embodiments, first side-camera 120b may be positioned at a distance of up to about 1.0 millimeters, up to about 5.0 millimeters, or up to about 15.0 millimeters from front surface 146. Each possibility corresponds to separate embodiments. According to some embodiments, second side-camera 120c may be positioned at a distance of up to about 1.0 millimeters, up to about 5.0 millimeters, up to about 15.0 millimeters, or up to about 25.0 millimeters from front surface 146, such as to optionally be positioned farther from front surface 146 than first-side-camera 120b. Each possibility corresponds to separate embodiments. Each possibility corresponds to separate embodiments. According to some embodiments, second side-camera 120c may be positioned at a distance of between about 1.0 millimeters to about 5.0 millimeters, about 5.0 millimeters to about 15.0 millimeters, or about 5.0 millimeters to about 25.0 millimeters from front surface 146, such as to optionally be positioned farther from front surface 146 than first-side-camera 120b. Each possibility corresponds to separate embodiments. According to some embodiments, the positioning of cameras 120 on shaft distal section 112 is selected such as to minimize the space occupied by cameras 120 and reduce the diameter of shaft distal section 112, while affording a continuous and consistent HFOV of at least about 270 degrees.

According to some embodiments, each of cameras 120 is associated with a respective illumination component from illumination components 122, which is configured to illuminate the FOV of the camera. Thus, according to some embodiments, front camera 120a may be associated with a respective front illumination component (not numbered), first side-camera 120b may be associated with a respective first side-illumination component, and second side-camera 120c may be associated with a respective second side-illumination component.

According to some embodiments, not depicted in the figures, cameras 120 include only two cameras, both of which are side cameras with fish eye lenses. In such embodiments, shaft distal section 112 may taper in the distal section, such that the cameras provide a continuous HFOV. According to some embodiments, not depicted in the figures, cameras 120 include only two cameras: a front camera and a side camera.

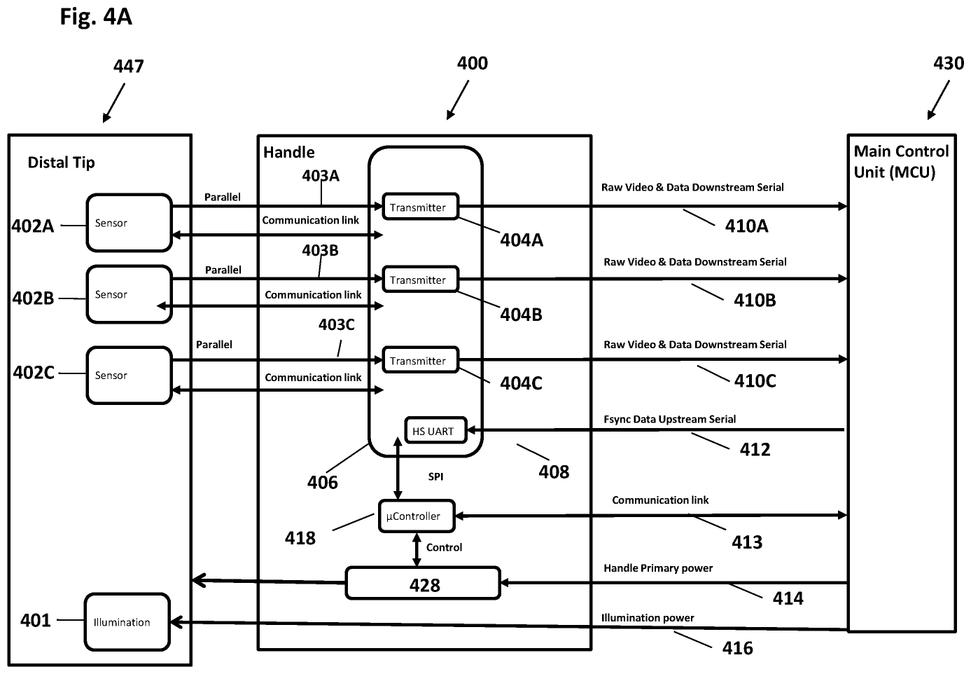

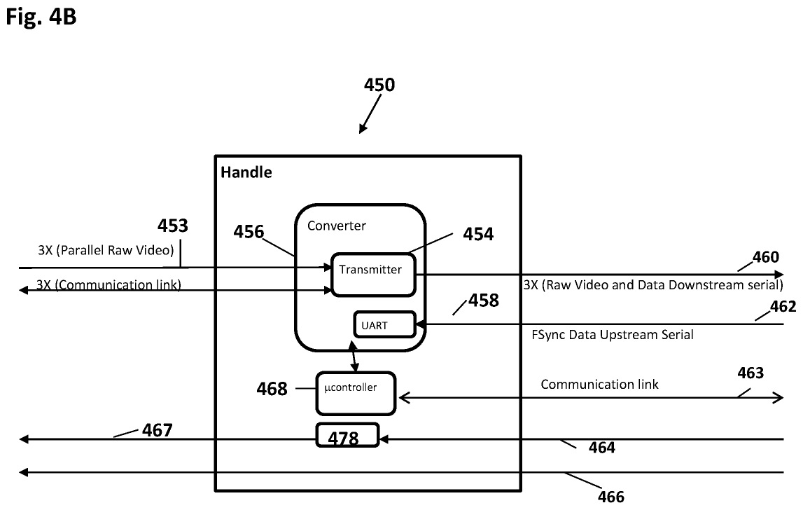

Reference is now made to FIG. 4A, which depicts a block diagram of components of an endoscope handle interacting with a main control unit and a distal tip of a medical imaging system, according to FIG. 2. As shown in FIG. 4A, handle 400 operatively connects with a distal tip 470 of a medical imaging endoscope and a main control unit 430. Distal tip 470 includes at least two sensors, in an embodiment shown in FIG. 4A distal tip 470 includes three sensors 402A, 402B, 402C, that receive information from corresponding lens assembly(s) located at the distal end 470, for example lens assembly of cameras 120 as disclosed in FIG. 1B to FIG. 3. The sensors may be selected from, for example, a charge coupled device (CCD) image sensor(s) or a complementary metal oxide semiconductor (CMOS) image sensor(s). The parallel signal/data generated by the sensors may then be transferred (for example, as parallel raw video data (403A-C)), to a converter 406. In some exemplary embodiments, the parallel raw video signal may be at 8-12 bit. In some exemplary embodiments, the parallel raw video signal may be at 9-11 bit. In some exemplary embodiments, the parallel raw video signal may be at 10 bit. Converter 406 is configured to convert the parallel signal into a serial signal. In some embodiments, converter 406 may be, for example, in the form of a FPGA that may further include various elements, including, for example, at least two transmitters, in an embodiment shown in FIG. 4A distal tip 470 includes three transmitters 404A, 404B, 404C corresponding to sensors 402A, 402B, 402C, respectively. Each sensor transfers in parallel signals a raw video downstream to a corresponding transmitter located within convertor 406. Each of the transmitters 404A, 404B, 404C is configured to transmit/relay the serial signal generated by the corresponding sensor converter 406 to a main control unit 430. A serial signal (shown as signals 410A-C), transmitted to main control unit 430 may include, for example, raw video and data signals. In some embodiments, the data signal may include such data as, but not limited to: data related to the operation of the endoscope (such as, activating buttons' status, internal registers, data extracted from the sensors' embedded data line (sensor temperature, gain, exposure time), and the like. In some embodiments, the data may be multiplexed with the video (for example, by arranging the data in packets to be multiplexed with the video packets). In some exemplary embodiments, the serial signal may be transmitted at a rate of about 1-2 Giga bits per second (Gbps), for example in the rate of about 1.5 Gbps, for example, 1.485 or 1.485/1.001 Gbps. The transmission of the serial signal may include any suitable protocol, using any suitable connection interface. The handle further includes additional electronic components, including UART element 408 (such as, for example, a High Speed UART), that may be connected to the main control unit 430 via suitable connecting interface (shown as interface 412). In some embodiments, the HS UART bit rate is an integer division of the control unit (Host) frequency and such rate selection can reduce the jitter of FSIN embedded message on that line. In some embodiments, interface 412 is configured to relay serial signals (for example at a rate in the range of from about 0.5 to 10 or to 12 Gbps) from main control unit 430 to UART 408 of controller 406. In some exemplary embodiments, interface 412 is an FSync data upstream serial configured to a unidirectional communication. The handle may further include a micro-controller such as, μcontroller 418. The micro-controller 418 may be connected/linked to the main control unit 430 via suitable connecting interface, link interface 413, and protocols. Micro-controller 418 may further include an input/output function configured for interaction with external (out of the system) devices, for example, when reprograming (update and/or change) the FPGA is required. In some embodiments, link interface 413 includes a serial interface/link. In some exemplary embodiments, link interface 413 includes link/interface, such as RS-485, RS232, RS422, USB, SPI, and the like, or any combination thereof. Link interface 413 may further be configured to control the sensors 402A-C and/or to control the handle 400. Handle 400 may further include a secondary power supply 428, configured to dispense/provide power to components of the handle 400 and/or distal tip 470. Secondary power supply 428 receives power from a primary power supply within the main control unit 430. Further, the main control unit 430 may provide direct power 416 to other components in the distal tip 470, such as, for example, to the illuminating sources (401), as detailed above herein. In some embodiments, direct power 416 provides power to the illuminating sources, for example for the LEDs located in distal tip 470. In an embodiment, direct power 416 includes three power resources for example, for three groups of LEDs, (for wherein the first power source is for a front LEDs group adapted to illuminate a front field of view corresponding to a front camera, a second power source is for a first side LEDs group adapted to illuminate a first side field of view corresponding to a first side camera, and third power source for a second side LEDs group adapted to illuminate a second side field of view corresponding to a second side camera. The power for each of the LEDs groups may depend on pre-defined processing input within main control unit 430, configured to provide clear view of a video stream received from each of the cameras, such the intensity of each group of illuminators may be change independently during a medical procedure. Thus, the various components, located within the handle operate in synchronized, efficient and timely manner, using suitable connecting protocols and interfaces, to convert raw parallel data obtained from one or more (for example, at least two) sensors, to a corresponding serial signal, and to relay that serial signal to a control unit for further processing (such as, displaying, etc., as detailed herein). In some embodiments, it is advantageous for the main control unit to supply various different voltages, that are regulated on the handle by means of linear regulators (LDO) instead of switched regulators.

Reference is now made to FIG. 4B, which depicts a block diagram of a handle board with a plurality of inputs and outputs, according to some embodiments. As shown in FIG. 4B, handle 450, receives data/information/signal from one or more sensors, located at a distal tip (not shown) of a medical imaging endoscope, for example medical imaging endoscope 100 depicts in FIG. 2. The sensors are of corresponding camera(s) located at the distal tip of the medical imaging endoscope. The sensors may include, for example, a charge coupled device (CCD) image sensor(s) or a complementary metal oxide semiconductor (CMOS) image sensor(s). A parallel signal/data generated by the sensors may then be transferred as parallel raw video data 453, to a converter 456, located in handle 450. The transfer of the data from the sensors to transmitters 454 may be performed using dedicated means, including, for example, wires or flexible PCB elements connected to the sensors located in the distal tip of a rigid/semi rigid shaft of the medical imaging endoscope, to the converter 456 located in the handle 450. In some exemplary embodiments, the parallel raw video signal may be at 10 bit. In some embodiments, the 10 bit includes raw pixel value plus H and V sync signals, accompanied by their related PCLK (peripheral clock). Converter 456 is configured to convert the received parallel signal into a serial signal. In some embodiments, the converter 456 may be, for example, in the form of a FPGA that may further include various elements, including, for example, one or more transmitters (shown as transmitter unit 454). The transmitters 454 are configured to transmit/relay the serial signal generated by the converter to an external control unit (not shown), for example to main control unit 430, according to FIG. 4A. The serial signal (shown as signal 460), transmitted/downstreamed to main control unit may include, for example, raw video and/or data signals. In some exemplary embodiments, the serial signal may be transmitted at a rate of about 1-2 Giga bits per second (Gbps), for example in the rate of about 1.5 Gbps. The transmission of the serial signal 460 may include any suitable protocol, using any suitable connection interface. The handle 450 further includes additional electronic components, including UART element 458 such as, for example, a High Speed UART, that may be integrated within convertor 456 and further may be connected to the main control unit via suitable connecting interface (shown as interface 462). In some embodiments, interface 462 is configured to relay serial signals. In some exemplary embodiments, interface 462 is an FSync data upstream serial. The handle 450 further includes a micro-controller (such as, μcontroller 468). The micro-controller 468 may be connected/linked to the main control unit via suitable connecting interface (link interface 463), and protocols. In some embodiments, link interface 463 includes a serial interface/link. In some exemplary embodiments, interface 463 includes RS-485 link. Further, the main control unit may provide direct power to handle 450 for controlling operation means within handle 450 and sensors. Handle 450 further includes a secondary power supply 478, configured to dispense/provide power to components of the handle 450 and/or the distal tip. Such components of handle 450 may include control means such as buttons, switches, knobs and the like, for controlling the endoscope functionality and operationally parameters such as, zoom, focus, record and the like. In some cases, the control means may also be utilized to change the layout of video configurations display on a monitor such as monitor 220 according to FIG. 2. Secondary power supply 478 receives power from a primary power supply in the main control unit. The main control unit may further provide direct power to other components in the distal tip of the medical imaging endoscope, such as, for example, to the illuminating sources (not shown). Thus, the various components, located within the handle 450 operate in synchronized, efficient and timely manner, using suitable connecting protocols and interfaces, to convert raw parallel data obtained from one or more sensors (at least two sensors), to a corresponding serial signal, and to relay that serial signal to main control unit for further processing (such as, displaying, etc., as detailed herein).

In some embodiments, the convertor 456 may receive parallel video signal 453 which, in one embodiment, comprises three video feeds corresponding to video data streams initiated by three sensors (shown in FIG. 3 as front camera 120a, first side camera 120b and second side camera 120c). The direct power (may also refer to sets of signals) may include individual three sets of signals for the front, first side and second side LEDs, respectively.

In other embodiments, not shown in any of FIGS. 3, 4A-4B, a handle of a medical imaging endoscope may include one or more optical sensors, that receive information from corresponding lens assembly(s) located at a distal tip of the medical imaging endoscope, for example medical imaging endoscope 100 depicts in FIG. 2. The sensors may be selected from, for example, a charge coupled device (CCD) image sensor(s) or a complementary metal oxide semiconductor (CMOS) image sensor(s). In some embodiments, the handle may include at least two optical sensors, that receive information from corresponding lens assembly(s) located at a distal tip of the medical imaging endoscope. In some embodiments, the handle may include at least three optical sensors, that receive information from corresponding lens assembly(s) located at a distal tip of the medical imaging endoscope.

In some embodiments, more than one sensor (for example, 1.5 or two sensors), may be serialized per a single serial stream. In such embodiments, prior to serialization, the FPGA may arrange the data in packets. Such packetization enables future multiplexing of more than one sensor per cable/communication means.

In some embodiments, the internal clock of the handle does not need to track (lock on) the clocks of the main control unit of the transferring and processing of the signals and/or data.

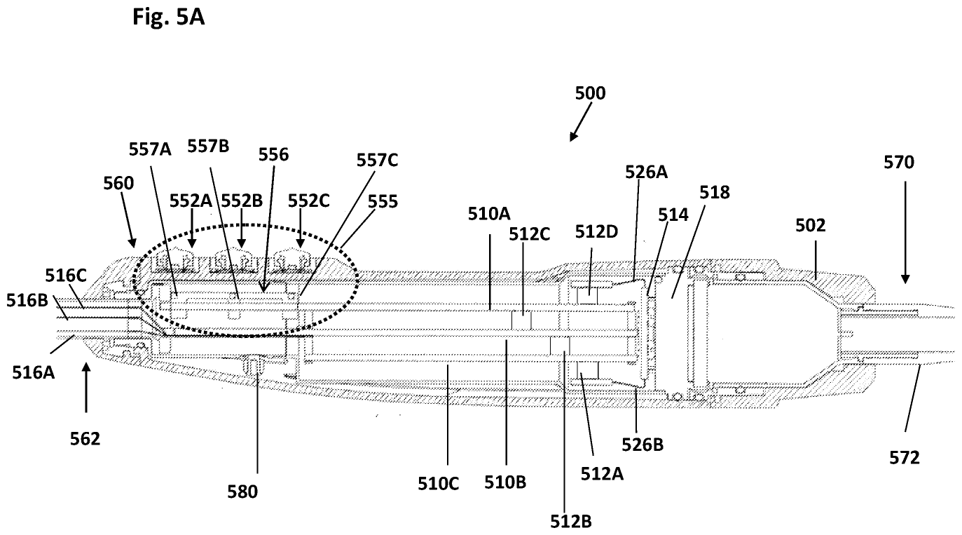

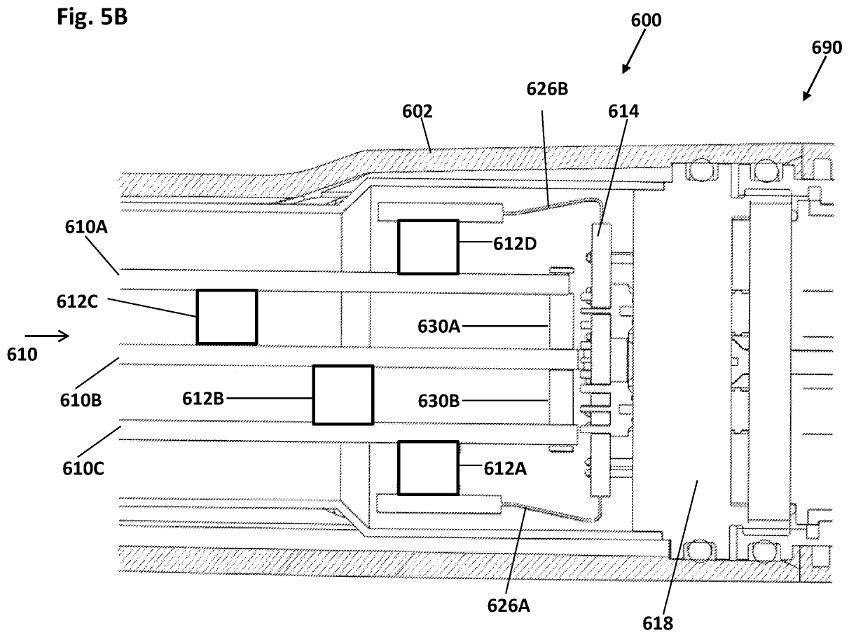

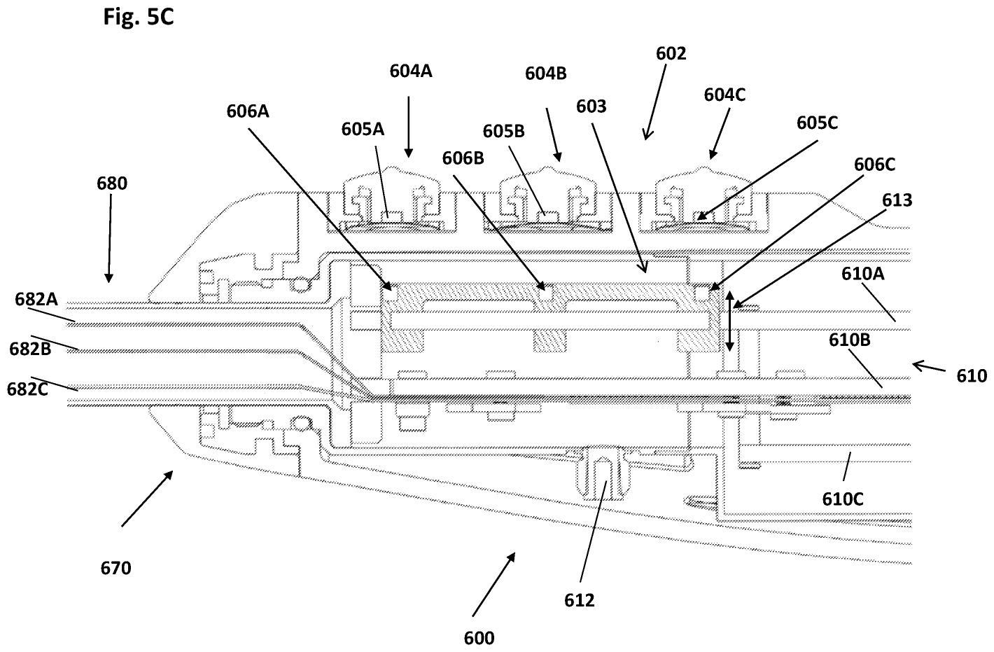

Reference is now made to FIG. 5A, depicting a schematic, perspective (partial) view of a cross sectional view of a handle, according to some embodiments. As shown in FIG. 5A, handle 500 has a distal end 560 and a proximal end 570. The handle 500 further includes an outer casing 502. The proximal end 570 is configured to connect to an external main control unit (not shown), via suitable connecting interface 572 that may include any suitable connectors, adaptors, linker, cables, wires, and the like. In another embodiment of the present invention, wireless communication between handle and main control unit can be configured and utilized. The distal end 560 is configured to connect to an elongated shaft; the proximal end of the shaft is shown, 562. A full assembly of handle 500 operationally incorporated at distal end 560 with the elongated shaft and at proximal end 570 with the main control unit is disclosed in FIG. 1B. From optical sensors located at a distal tip of the shaft (not shown), signals (such as parallel signals) are transferred to the handle 500, via suitable transferring means/elements (shown as transferring means 516A-C, obtained from three optical sensors). The transferring means may include any compatible transferring means, such as, for example, cables or flexible PCBs or rigid PCBs or flex-rigid PCBs or any combination therefore. Each possibility is a separate embodiment. The transferring means may be located within the shaft of the endoscope and run along its longitudinal axis. The signals (such as parallel signals) transferred to the handle 500 are conveyed via suitable PCB elements 516A-C, to a converter 510. As shown in FIG. 4A, the converter 510 may include one or more PCBs, 510A-C, arranged in parallel to each other, and to a longitudinal axis of the handle 500. The converter PCBs 510A-C may be identical, similar or different with respect to size, relative position, composition and/or function. In some exemplary embodiments, the converter PCBs 510A-C may be rigid or semi rigid. In some embodiments, one of the converter PCBs (for example, the middle converter PCB, 510B) may be configured to transmit the signals (such as parallel signals) obtained from the optical sensors to the converter 510. In some exemplary embodiments, the top converter PCB 510A may be connected to a user interface activating unit (such as user control interface 138 of FIG. 1B) of the handle 500, or user interface activating unit may be connected thereto. The user interface activating unit (marked by circle 555) includes external control buttons (shown as external activating buttons, 552A-C), and an internal interface in the form of internal activating element/unit (556). The internal activating element 556 is shown as an elongated board, located/mounted on a PCB (for example, top converter PCB, 510A) and further includes sensors (shown as sensors 557A-C), configured to interface/interact with the corresponding external activating buttons 552A-C. In some embodiments, the sensors 557A-C face a lower surface of the external activating buttons 552A-C. In some embodiments, the sensors 557A-C and the external activating buttons 552A-C do not physically contact each other. In some embodiments, the external activating buttons 552A-C may be located close to the distal end 560 of the handle 500, and are configured to allow/mediate controlling the operation of the handle 500 by the user, by interfacing with the internal activating element 556, for example, by interfacing with the respective sensors 557A-C, as further detailed below. In some embodiments, the top converter PCB 510A may interact with the user interface activating unit (or at least some components thereof) via magnetic means. In some exemplary embodiments, the lower converter PCB 510C may be configured to interact with various power supplies and/or microcontrollers. The converter 510 may be any type of suitable converter configured to convert the signals (for example, parallel signals) conveyed from the sensors, via, for example, PCB elements, to suitable signals (such as, serial signals) and to further relay that signal via suitable interfaces and protocols (as detailed above) to the main control unit placed externally to handle 500.

As shown in FIG. 5A, the handle 500 further includes one or more connectors, such as electrical connector elements 512A-D, that provide mechanical support for the placement, anchoring and/or orientation of the various converter PCBs (510A-C), as well as allow interconnection between the various converter PCBs 510A-C themselves and/or between the PCB elements and a connector 518. The electrical connector elements 512A-D may be rigid, semi-rigid, flexible, or any combination thereof. The electrical connector elements 512A-D may be identical, similar or different from each other with respect to: size, composition, orientation, location, function and/or structure. In some embodiments, by the advantageous setting of the converter PCBs 510A-C within the limited space of the handle 500, allows the handle to accommodate all electronic and mechanical elements required to achieve the goal of converting a raw parallel signal to a raw serial signal within the handle, and to further successfully rely the raw video serial signal and data downstream to the main control unit for further manipulation. In some embodiments, the advantageous setting of the internal units of the handle allows smooth and accurate operation of the endoscope, while maintaining the integrity and sealing of the handle and without generating excessive internal heat.

According to some embodiments, handle 500 further comprise a round PCB 514. Round PCB 514 may be a rigid PCB, a semi rigid PCB, or a flexible PCB. In some embodiments, round PCB 514 distal end may include two flexible arms/PCBs 526A and 526B. Flexible arm/PCB 526A is configured to electronically connect to converter PCB 510A with electrical connector 512D, and flexible arm/PCB 526B is configured to electronically connect to converter PCB 510C with electrical connector 512A. Round PCB 514 may be further configured to mechanically encore convertor PCB 510B. Round PCB 514 proximal end is connected to connector 518, wherein connector 518 is further connected to the connecting interface 572. Signals produced at the distal tip can relay/travel on PCBs 516 into converter 510 further away to round PCB 514, which is part of connector 518, to the main control unit. The main control unit may perform interpolation processes on the raw video streams (for example, raw serial streams) to generate a video configuration to be displayed on a monitor.

In some embodiments, outer casing 502 may be configured to fit over the internal components of handle 500, including converter 510 and internal activating element/unit 556, and other electronic components as disclosed herein, and to provide protection to the internal components, which prevents leaking of liquids, gases and/or debris or body fluids into inner parts of handle 500. According to some embodiments, the outer casting 502 may be made of materials configured to prevent fluid and debris from entering the handle chamber and also may also be configured to withstand various sterilization processes, such as, for example, autoclave sterilization. In some exemplary embodiments, the outer casing may be made of plastic and/or silicon.

In some embodiments, the connection between the proximal end of the shaft 562 and the outer casting 502 may be achieved by an adhesive material that seals the connection. In some other embodiments, the proximal end of the shaft 562 and the outer casting 502 may be connected by soldering. In some embodiments, the proximal end of the shaft 562 and the outer casting 502 may be connected by a screwing mechanism which fastens the proximal end of the shaft 562 and the outer casting 502 together.

In such embodiments, the connection between the connecting interface 572 and the outer casting 502 may be by an adhesive material that seals the connection. In some other embodiments, the connecting interface 572 may be connected by soldering. In possible embodiments of the disclosed subject matter, the connecting interface 572 may be connected by a screwing mechanism which fastens the connecting interface 572.

In some embodiments, a leak tester 580 may be placed within distal end 560 of handle 500. Leak tester 580 may be operated prior to sealing handle 500 with outer casting 502. A leaking test allows maintaining a sealed environment within handle 500, such ensure preventing fluids, liquids and or gas, from entering the internal regions of the handle and effect the efficiencies of internal components, internal components such as PCBs, converter, connector and others electronical and functional components/elements.

Reference is now made to FIG. 5B, which depicts a schematic, perspective close-up view of a cross section of a proximal end region of an endoscope handle, according to that shown in FIG. 5A. As shown in FIG. 5B, a proximal end 690 of a handle 600 includes a converter (610), mounted essentially parallel to the longitudinal axis of the handle 600. The converter may include one or more PCBs, shown as PCBs 610A-C arranged in parallel to each other, and to the longitudinal axis of handle 500. converter 610 further includes one or more connectors such as connector elements 612A-D, that allow interconnection between the various converter PCBs 610A-C themselves and/or between the PCB elements and a connector 618. The electrical connector elements 612A-D may be rigid, semi-rigid, flexible, or any combination thereof. The electrical connector elements 612A-D may be identical, similar or different from each other with respect to: size, composition, orientation, location, function and/or structure Proximal end 690 further includes connecting elements, which can be in some examples, PCB units. In some embodiments, a PCB unit such round PCB 614 may be used to electronically connect/couple the handle electronic elements to a proximal connector 618, configured to allow connection (physically and/or functionally) of the converter with the external main control unit (not shown), via suitable downstream connecting interfaces, that may include any suitable connectors, adaptors, linker, cables, wires, and the like. The round PCB 614 (which may be rigid, semi-rigid or flexible) can further provide mechanical support to the converter 610 and/or other PCB elements. In some embodiments, as detailed above, the converter 610 may be a FPGA. Further shown in FIG. 5B, are the proximal ends of converter rigid PCB elements (610A-C), which are placed essentially in parallel to the longitudinal axis of the handle, as detailed above. The handle further includes, in some exemplary embodiments depicted in FIG. 5A, flexible arms/connectors/PCBs, such as connectors/PCBs 626A-626B, configured to functionally connect the parallel PCBs 610A and 610C, respectively, to the round connector 614. Mechanical/mounting elements 630A-B are configured to provide mechanical support for the placement, encore and orientation of the various converter PCBs 610A-C. Further shown in FIG. 5B, is cross section of the handle casing/cover 602, which allows a sealed internal space within the handle.

Reference is now made to FIG. 5C, which depicts a schematic, perspective close-up view of a cross section of a distal end region of an endoscope handle, according to that shown in FIG. 5A. As shown in FIG. 5C, a distal end 670 of a handle 600 includes a user interface activating unit 602, which includes external activating/control buttons (shown as external activating/control buttons, 604A-C), and internal activating element/unit 603. The internal activating element 603 is an elongated board, on the face of which, sensors (such as sensors 606A-C), which are configured to interact with the external activating buttons 604A-C. In some embodiments, as shown in FIG. 5C, on the lower surface of the activating buttons 604A-C (i.e., the surface that faces the internal activating element/unit 603), magnets, (such as, magnets 605A-C) are placed/located. Additionally, or alternatively, in some embodiments, a magnet is not necessarily present, but rather, at least a portion of the lower surface of the external activating buttons 604A-C, is magnetized or has magnetic properties. The sensors 606 of the internal activating element 603 can interface with the external activating buttons 604, to exert operating commands from the external activating buttons to the internal space of the handle, via the sensors, to allow control and operation of the handle 600 and hence of the endoscope. In some embodiments, the sensors 606 are magnetic sensors, interacting/interfacing with the magnet (or magnetized region) of the external activating buttons 604. In some embodiments, the sensors 606 are linear sensors. In some embodiments, the sensors 606 are analog sensors. In some embodiments, the sensors 606 may include a mixture of analog and linear sensors. In some embodiments, distance and/or voltage difference between the sensors 606 and the external activating buttons 604 may be predetermined when fabricating the handle 600. In some embodiments, the external activating buttons 604 and sensors 606 do not physically contact each other. By such mode of operation, activation and control of the endoscope, is achieved, as detailed bellow.

The handle further includes a converter 610 mounted essentially parallel to a longitudinal axis of the handle 600. The converter 610 may include one or more PCBs, shown as PCBs 610A-C arranged in parallel to each other, and to the longitudinal axis of handle 600. Converter 610 was described in FIG. 5A with reference to converter 510. As shown in FIG. 5B, distal ends of converter rigid PCB elements (610A-C), are placed essentially in parallel to the longitudinal axis of the handle, as detailed above. Further shown in leak tester 612.

As further shown in FIG. 5C, the top converter PCB 610A is used to mount the internal activating element/unit 603. In some embodiments, the internal activating unit 603 may be rigid, semi rigid or flexible. In some embodiments, the internal activating unit 603 which includes sensors 606 is a PCB. In some embodiment, the internal activating unit 603 may be located/mounted/placed within the top converter PCB 610A. In some embodiments, the internal activating unit 603 may be movable, such as to allow adjusting the location of the sensors 606 of internal activating unit 603 with respect to magnets 605 of lower surface of activating buttons 604, prior to sealing the handle. By adjusting the location of the sensors 606, the most accurate and smooth interfacing between the external activating buttons 604 and the internal activating element/unit 603 is achieved, resulting in reliable, smooth and accurate operation of the handle 600. In some embodiments, adjusting the location relates to sideways movement of the internal activating element/unit 603, sideways movement which moves internal activating element/unit 603 along the longitudinal axis of converter PCB 610A, to align the sensors 606 with their corresponding interfacing region of the external activating buttons 604. In some embodiments, adjusting the location relates to adjusting the height (distance) between the internal activating element/unit 603 and the lower surface of the external activating buttons 604, the adjusting move may be in a perpendicular direction 613 to the longitudinal axis of top converter PCB 610A, such as to ensure the most accurate interfacing between the sensors 606 and their respective counterpart region of the external activating buttons 604. In some embodiments, once the desired location of the internal activating element/unit 603, it can be fixed (for example, by welding) to the PCB.

Further shown in FIG. 5C is the proximal end of rigid shaft, 680, which connects to the distal end 670 of the handle 600. Also shown are suitable transferring means (shown as transferring means 682A-C), configured to convey signals, data, power, and the like, to and from the distal end of the shaft (not shown). The transferring means 682A-C may include any compatible transferring means, such as, for example, cables, wires, flexible PCBs, rigid PCBs, flex-rigid PCBs, and the like, or any combination therefore. Each possibility is a separate embodiment. The transferring means may be located within the shaft of the endoscope and run along its longitudinal axis, for example transferring means 516 as described in connection with FIG. 5A.

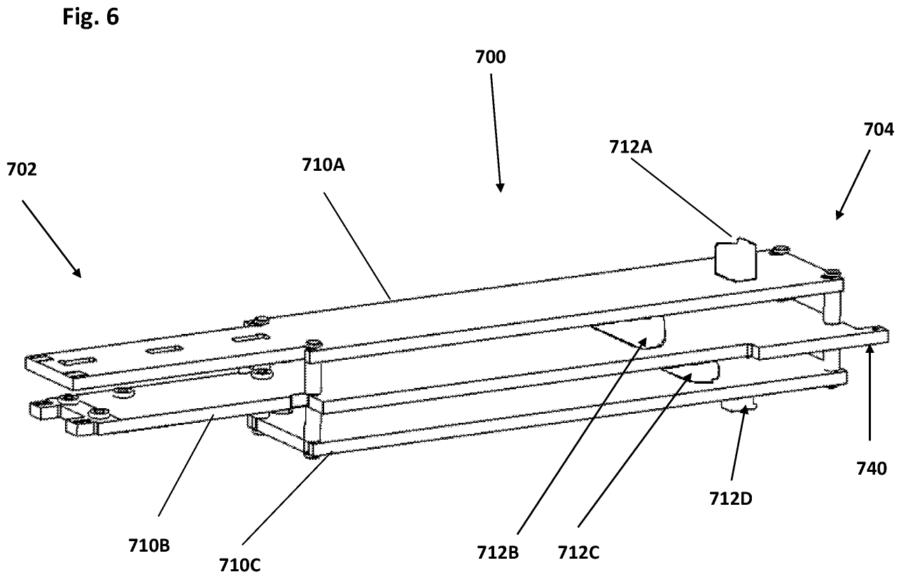

Reference is now made to FIG. 6, which depicts a schematic perspective view of settings of rigid PCB configured to be placed in a handle of an endoscope, with respect of that shown in FIGS. 5A-C, according to some embodiments. Shown in FIG. 6 are the architectural (physical) setting 700 of an array of a converter 710 which includes three PCB elements, 710A-C, of a handle (not shown) of a medical device. As shown, the three PCB elements 710A-C are located essentially in parallel to each other and to a longitudinal axis of the handle (not shown). The three PCB elements 710A-C extend from a distal end 702 to a proximal end 704. The three PCB elements 710A-C may be placed at similar or different vertical distance/s (space/s) from each other. The three PCB elements 710A-C may be rigid, semi rigid, flexible, or combinations thereof. The three PCB elements 710A-C may be identical, similar or different from each other with respect to: size, composition, orientation, location, function and/or structure. In some embodiments, each PCB of the three PCBs 710A-C may be connected to different electronical or mechanical elements. In some embodiments, the three PCB elements 710A-C may be at least partially interconnected physically and/or functionally, either directly or via connecting elements/connectors.

Further shown in FIG. 6 are mechanical/mounting elements 730A-730B, which are configured to assist in the physical mounting of the three PCB elements 710A-C, for example, by providing mechanical support, maintaining the distance between the three PCB elements 710A-C, and the like. The mechanical/mounting elements 730A-730B may be made of any suitable material and may be conductive or non-conductive. The mechanical/mounting elements 730A-730B may be identical, similar or different from each other, with respect to size, structure, location, composition and/or function. In some embodiments, electrical connectors, shown as electrical connector elements 712A-D, are configured to allow electrical, functional and/or physical connection between the various PCB elements 710A-C therebetween, or between the PCB elements and other functional elements of the handle (such as round PCB and connector, for example round PCB 514, 614 and connector 518, 618 according to FIGS. 5A, 5B, respectively). The electrical connectors 712A-D may be rigid, semi-rigid, flexible, or any combination thereof. The electrical connectors 712A-D may be identical, similar or different from each other, with respect to size, structure, location, composition and/or function.

In some embodiments, the three PCB elements 710A-C may include additional features (mechanical or electronical) to aid in their functioning. For example, as illustrated in FIG. 6, top converter PCB 710A may include grooves 740A, 740B, 740C upend within distal end 702 thereof, to accommodate the internal activating element of the user interface activating unit, as detailed above. For example, a user interface activating unit 555, 602 of handle 500, 600, and internal activating element/unit 556, 603 according to FIGS. 5A and 5C, respectively. The three grooves 740A-C may be placed at similar or different distance/s (space/s) from each other. The three grooves 740A-C may be identical, similar or different from each other with respect to: size, orientation, location, function and/or structure. The internal activating element may have corresponding hinges/prongs or other means/elements to fit into the grooves, so as to ensure accommodation (with respect of secured holding and correct placement) of the internal activating element.

Furthermore, as illustrated in FIG. 6, middle converter PCB 710B, may include anchoring components configured to anchor converter 710 in a pre-defined place/location within the handle. Due to space constrains within the handle and the number of components connected to the converter three PCBs 710A-C (such as, connectors 712A-D, internal activating element/unit of user interface activating unit, round PCB (for example round PCB 514 of FIG. 5A)) it is important to situate/position converter 710A-C in the pre-defined place to maintain signal quality conveyed from optical sensors and illuminators at a distal tip of the endoscope to a main control unit and the upstream commands from the main control unit to handle and into the distal tip. The anchoring components may be located at the proximal end 704 of middle converter PCB 710B, such as proximal hinge 760 at proximal end 704 thereof, to allow converter 710 positioning and securing within the handle. Proximal hinge 760 may include one or more hinges. Proximal hinge 760 may secure converter 710 within round PCB of the connector (for example round PCB 614 and connector 618 as illustrated in FIG. 5B). Middle converter PCB 710B, may further include one or more distal hinge 762 at distal end 702 of setting 700 of the handle. Distal hinges 762A-B may secure converter 710 structure within distal end 702. Distal hinges 762A-B may be identical, similar or different from each other, with respect to size, structure, location, composition and/or function. Top converter PCB 710A may also include anchoring components, such as, for example, distal hinge 764 configured to secure converter 710 structure within distal end 702.

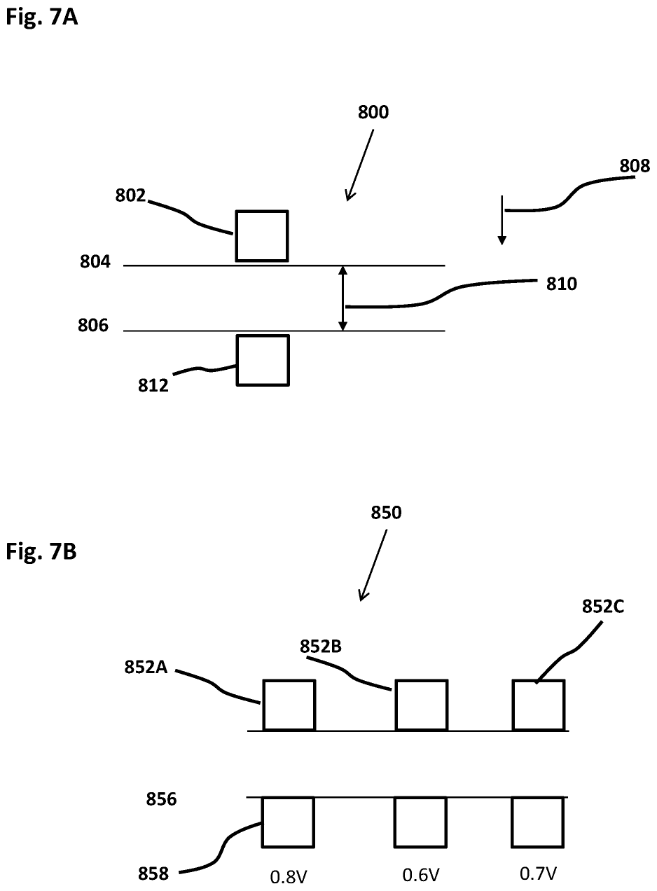

Reference is now made to FIGS. 7A-7B, which show schematic block diagrams of mode of operation of a user interface activating unit of a handle, according to some embodiments. Shown in FIG. 7A, is mode of operation of user interface activating unit which utilizes an analog magnetic sensor. As shown in FIG. 7A, user interface activating unit (800) includes external activating/control buttons (shown as exemplary activating button including a magnet or magnetic property, (802) and internal activating element/unit including an analog magnetic sensor 812. The user interface activating unit 800 may be part of a medical device endoscope handle, for example user interface activating unit (555, 602) of handle (500, 600) having an external activating/control buttons (552, 604) includes magnets, or magnetic properties, (605) and an internal activating element/unit (556, 603) includes sensors (606) as described with reference to FIGS. 5A and 5C, respectively. The activating button 802 can be at an “off” state 804 i.e, not interfacing with the corresponding analog magnetic sensor 812, or at “on” state 806 i.e., interfacing with the corresponding analog magnetic sensor 812, the difference between the on-off states is determined based on a difference in height/distance between the activating button 802 and the analog magnetic sensor 812.

Thus, when the activating button 802 is pressed (in the direction of arrow 808), for example, by a user, activating button 802 changes its position from “on” position at state 804 to “off” position at state 806 such, the height/distance difference 810 between the lower (magnetic) surface of the activating button 802 and the corresponding analog magnetic sensor 812 is changed, such as to be decreased/reduced, to result in switching to an “on” mode, to result in transferring the activation command to the handle, for further processing, by the handle itself, and/or by a main control unit, for example main control unit 210 as depicted in FIG. 2. In some embodiments, the height difference may be in the range of about 0.1-0.9 mm, or any subrange thereof. In some embodiments, the height difference may be in the range of about 0.3-0.8 mm. In some embodiments, the height difference may be in the range of about 0.5-0.7 mm. In some embodiments, the distance between the lower surface of the external activating button and the internal sensor at an “off state” may be in the range of about 1-9 mm. In some embodiments, the distance difference 810 between the lower surface of the external activating button 802 and the internal analog magnetic sensor 812 at an “off state” may be in the range of about 1-9 mm. In some embodiments, the distance difference 810 between the lower surface of the external activating button 802 and the internal analog magnetic sensor 812 at an “off state” may be in the range of about 3-8 mm. In some embodiments, the distance difference 810 between the lower surface of the external activating button 802 and the internal analog magnetic sensor 812 at an “off state” may be in the range of about 5-7 mm. In some embodiments, the distance difference 810 between the lower surface of the external activating button 802 and the internal analog magnetic sensor 812 at an “off state” may be in the range of about 0.01-1 mm. In some embodiments, the distance difference 810 between the lower surface of the external activating button 802 and the internal analog magnetic sensor 812 at an “off state” may be in the range of about 0.03-0.08 mm. In some embodiments, the distance difference 810 between the lower surface of the external activating button 802 and the internal analog magnetic sensor 812 at an “off state” may be in the range of about 0.05-0.07 mm.

In some embodiments, the activating button 802 stays “on” at state 806 until the user has re-pressed activating button 802 (in the direction of arrow 808) and activating button 802 returns to “off” state 804. In other embodiments, the activating button 802 stays “on” at state 806 as long as the user presses activating button 802.

It will be understood by those skilled in the art that the user interface activating unit may be calibrated during the assembly of the handle, so as to provide the required distance in order to switch activating button 802 from “off” state 804 to “on” state 806. In some embodiments, the calibration may be performed at one or more time points prior to assembling or sealing the handle. In some embodiments, the calibration ensures the most accurate, smooth and efficient operation of the control unit. In some embodiments, the calibration may be performed individually for each handle while the handle is being fabricated.

Reference is now made to FIG. 7B, which shows mode of operation of a user interface activating unit which utilizes linear magnetic sensor. As shown in FIG. 7B, user interface activating unit (850) includes external activating/control buttons (shown as external activating buttons, 852A-C). The user interface activating unit 850 may be part of a medical device endoscope handle, for example user interface activating unit (555, 602) of handle (500, 600) having an external activating/control buttons (552, 604) including magnets, or magnetic properties (605) and an internal activating element/unit (556, 603) including sensors (606) as described with reference to FIGS. 5A and 5C, respectively. The activating button 852 can be at an “on” state (i.e, interfacing with the corresponding internal magnetic sensor), or at “off” state (856), i.e., not interfacing with the corresponding internal magnetic sensor), the difference between the on-off states is determined based on the difference in voltage. Thus, when the external button is pressed, a change in voltage (i.e., a delta/change in voltage) is detected by the corresponding sensor, to result in switching to an “on” mode, to result in transferring the activation command to the handle, for further processing, by the handle itself, and/or by a main control unit (such as, for example main control unit 210 as depicted in FIG. 2). In some embodiments, the voltage difference may be in the range of about 0.1-0.7V, or any subrange thereof. In some embodiments, the voltage difference may be in the range of about 0.2-0.6 mm. In some embodiments, the voltage difference may be in the range of about 0.3-0.5 mm. In some exemplary embodiments, voltage difference may be about 0.3V. In some embodiments, the voltage at an “off state” may be in the range of about 0.4-1V. In other embodiments, the voltage at an “off state” may be in the range of about 0.5-0.9V. Yet in other embodiments, the voltage at an “off state” may be in the range of about 0.6-0.8.

Thus, when a change (delta) is detected by a sensor of an internal activating unit 858 the sensor (i.e., electromagnetic sensor), the corresponding operational instructions (operating commend) may be conveyed from the sensors to the corresponding suitable means (for example, wires, PCBs, FPGAs, processors, etc. that can convey the signal to the suitable element to perform the requested command). In some embodiments, such operational instructions may include such instructions as, but not limited to: operation of cameras at the distal tip of the shaft, including, for example, image or video recording, zoom-in, zoom-out, capture, freezing an image; controlling additional components in the distal shaft, including, for example, changing intensity of illumination component(s), changing frequency of illumination components, changing color of illumination component(s), and the like, or any combination thereof. Each possibility is a separate embodiment.

According to some embodiments, the internal activating unit may include one or more sensors. In some embodiments, the internal activating unit may include at least one sensor. In some embodiments, the internal activating unit may include at least two sensors. In some embodiments, the internal activating unit may include at least three sensors. In some embodiments, the number of sensors is determined based on the number of the external activation/control buttons. In some embodiments, the sensors may be identical or similar with respect to size, shape, structure, composition and/or function. Each possibility is a separate embodiment. In some embodiments, the sensors elements may be different from each other with respect to size, shape, structure, composition and/or function. Each possibility is a separate embodiment.

According to some embodiments, the external activating buttons and the internal sensors do not physically interact, i.e, there is at least a minimal distance there between, such that they do not touch each other. According to some embodiments, such setting is in particular advantageous, as it allows full/complete sealing of the handle on the one hand and further allow smooth, accurate and sensitive control and operation of the handle by a user, thanks to the advantageous user interface activating unit.

According to some embodiments, as detailed above, the relative location of the external activating buttons and the internal activation element is adjusted to ensure optimal magnetic field between the external magnetic control buttons and the internal electro magnetic sensors, to results in maximal sensitivity, accuracy and ease of operation.

According to some embodiments, the handle may include one or more PCB elements. In some embodiments, the handle may include at least one PCB element. In some embodiments, the handle may include at least two PCB elements. In some embodiments, the handle may include at least three PCB elements. In some embodiments, the handle may include at least two or more PCB elements. In some embodiments, the handle may include three or more PCB elements. In some embodiments, the PCB elements may be identical or similar with respect to size, shape, structure, composition and/or function. Each possibility is a separate embodiment. In some embodiments, the PCB elements may be different from each other with respect to size, shape, structure, composition and/or function. Each possibility is a separate embodiment. In some embodiments, at least one of the PCB elements is placed in parallel to the longitudinal axis of the handle. In some embodiments, at least one of the PCB elements is placed horizontally relative to the longitudinal axis of the handle. In some embodiments, at least one PCB element may extend (run) along the longitudinal axis of the handle, from a distal end to a proximal end.

According to some embodiments, there is provided an endoscope which includes the advantageous handle as disclosed herein, having the advantageous user interface activating unit, and a compatible shaft. In some embodiments, the shaft is elongated.

According to some embodiments, there is provided an endoscope which includes the advantageous handle as disclosed herein, which allows conversion of parallel signal to a serial signal, within the handle, and a compatible shaft. In some embodiments, the shaft is elongated.

According to some embodiments, there is provided a method for maintaining signal quality conveyed by one or more parallel signals output by one or camera sensors of an endoscope, the method comprising converting the parallel signals to serial signals in a handle of the endoscope and conveying said serial signals to an external control unit, functionally associated with the handle.

According to some embodiments, there is provided a method for preventing degradation of a signal conveyed by one or more parallel signals output by one or camera sensors of an endoscope, the method comprising converting the parallel signals to serial signals in a handle of the endoscope and conveying said serial signals to an external control unit, functionally associated with the handle.

According to some embodiments, there is provided a method for limiting degradation information conveyed by one or more parallel signals output by one or more cameras of an endoscope, the method comprising converting the parallel signals to serial signals in a handle of the endoscope and conveying said serial signals to an external control unit, functionally associated with the handle.

According to some embodiments, there is provided a method of converting a parallel signal output from one or more cameras of an endoscope, to a serial signal, and relaying said serial signal to a remote location, wherein the conversion is performed in a handle of the endoscope. According to some embodiments, the parallel signal is obtained at a distal end of the endoscope and converted to a serial signal in the handle.

In some embodiments, the serial signal generated/converted in the handle of the endoscope is relayed to an external control unit, functionally associated with the distal end of the handle.

According to some embodiments, there is provided a handle of a multi-camera endoscope, the handle includes a conversion unit/converter configured to receive one or more parallel signals and transform said one or more parallel signals to one or more corresponding serial signals, wherein the parallel signals are output by one or more cameras of the endoscope, said conversion unit/converter is further configured to transmit or relay the serial signals to an external control unit.

According to some embodiments, the conversion unit/converter may be configured to relay the serial signals to a utility cable connected to the external control unit that is functionally associated with the handle.

According to some embodiments, the external control unit may be connected to a proximal end of the handle. In some embodiments, the external control unit may further include one or more of: a user interface, a display, a power source, a communication unit, or any combination thereof. Each possibility is a separate embodiment.

According to some embodiments, the sensors may be located at a distal section of a shaft of the endoscope, the shaft being mounted on the distal end of the handle. According to some embodiments, the sensors may be located in the handle.

According to some embodiments, the transmitting to the control unit may be direct or via one or more intermediate elements, such as various communication units/elements, controllers, UART, and the like, or any combination thereof. Each possibility is a separate embodiment. In some embodiments, the transmitting is wired, using suitable communication wires, cables and protocols. In some embodiments, the transmitting is wireless, using suitable wireless transmitters/communication elements and protocols.

According to some embodiments, the conversion unit/converter may include a Field Programmable Gate Array (FPGA).

According to some embodiments, the handle may include one or more Printed Circuit board(s) (PCBs), communicatively associated with the sensors.

According to some embodiments, the handle may include at least two PCBs. In some embodiments, the handle may include at least three PCBs. In some embodiments, the at least two PCBs may include rigid PCB, flexible PCB, or both. In some embodiments, at least two PCB's are situated/mounted essentially in parallel to each other, along a longitudinal axis of the handle. In some embodiments, at least three PCB's are situated in parallel to each other, along a longitudinal axis of the handle.

According to some embodiments, the conversion unit/converter may be mounted on a PCB. In some embodiments, the PCB on which the conversion unit/converter is mounted/placed/located/associated with, may be placed perpendicularly to the longitudinal axis of the handle.

According to some embodiments, each of the sensors may be connected to a flexible or rigid PCB or to suitable wire/cable, extending along the shaft of the endoscope and connected to the one or more PCBs located in the handle.

According to some embodiments, the handle may further include a user control interface, including one or more operating buttons. According to some embodiments, the user control interface is functionally associated with the one or more handle PCBs, allowing control over operation of the endoscope. In some exemplary embodiments, the operating buttons may be magnetically operated.

According to some embodiments, the handle may further include one or more secondary power sources (power supply), configured to receive power from a primary external power source and distribute power within the handle.

According to some embodiments, the handle may further include one or more of: a main FPGA, a micro-controller (μcontroller), a universal asynchronous receiver-transmitter (UART), a mounting element, a connecting element, a secondary FPGA, or any combination thereof.

According to some embodiments, there is provided an endoscope which includes the handle, as disclosed herein, and an elongated compatible shaft.

According to some embodiments, the endoscope may include a plurality of cameras positioned at a distal section of the shaft. In some embodiments, the cameras may provide combined and consistent panoramic view is obtained from the cameras.

According to some embodiments, the endoscope may include at least one camera and at least one illumination component located at the shaft distal section.

According to some embodiments, the endoscope may include at least two cameras.

According to some embodiments, each of the sensors may be connected to a flexible PCB, or a suitable transmitting wire, extending along the shaft of the endoscope and connected to the one or more PCBs located in the handle.

According to some embodiments, the at least two cameras of the endoscope may include a front camera on a distal tip of the shaft and a first side-camera. According to some embodiments, the at least two cameras may include a second side-camera, wherein the first side-camera and the second side-camera are positioned on opposite sides of the shaft, and wherein the first side-camera is positioned distally relative to the second side-camera.

According to some embodiments, the at least two cameras may provide at least about 270 degrees horizontal field-of-view (FOV) of a target area within an anatomical cavity into which the elongated shaft is inserted.

According to some embodiments, the at least one illumination component may be a discrete light source, such as, for example, a light emitting diode (LED).

According to some embodiments, there is provided a method for maintaining signal quality and/or for preventing degradation of a signal and/or for limiting degradation information conveyed by parallel signals output by at least two cameras of an endoscope, the method comprising converting the parallel signals to serial signals in a handle of the endoscope and conveying said serial signals to an external control unit, functionally associated with the handle.

According to some embodiments, there is provided a method of converting a parallel signal output from at least two cameras of an endoscope, to a serial signal, and relaying said serial signal to a remote location, wherein the conversion is performed in a handle of the endoscope. According to some embodiments, the parallel signal is obtained at a distal end of the endoscope and converted to a serial signal in the handle. In some embodiments, the serial signal generated/converted in the handle of the endoscope is relayed to an external control unit, functionally associated with the distal end of the handle.

According to some embodiments, there is provided a handle of a multi-camera endoscope, the handle includes a converter configured to receive one or more parallel signals and transform said one or more parallel signals to one or more corresponding serial signals, wherein the parallel signals are output by one or more sensors of the endoscope, said converter is further configured to transmit or relay the serial signals to an external control unit.

According to some embodiments, the converter may be configured to relay the serial signals to a utility cable connected to the external control unit that is functionally associated with the handle.

According to some embodiments, the external control unit is connected to a proximal end of the handle.

According to some embodiments, the one or more sensors may be located at a distal section of a shaft of the endoscope and wherein each of the one or more sensors is associated with a lens assembly to obtain a camera, the shaft being mounted on the distal end of the handle.

According to some embodiments, the sensors may be located in the handle and each of the one or more sensors is associated with a lens assembly located at distal section to obtain a camera, the shaft being mounted on the distal end of the handle.

According to some embodiments, the transmitting to the control unit may be direct or via one or more intermediate elements.

According to some embodiments, the converter includes or is a Field Programmable Gate Array (FPGA).

According to some embodiments, the handle may include one or more Printed Circuit board(s) (PCBs), communicatively associated with the sensors. According to some embodiments, the handle may include at least two PCBs. According to some embodiments, the one or more PCBs may include rigid PCB, flexible PCB, or both. According to some embodiments, at least two PCB's may be placed parallel along a longitudinal axis of the handle. According to some embodiments, the handle may include at least three PCBs.

According to some embodiments, the converter may be mounted on a PCB.

According to some embodiments, each of the one or more sensors may be connected to a flexible PCB, extending along the shaft of the endoscope and connected to the one or more PCBs located in the handle.

According to some embodiments, the handle may further include a user control interface, comprising one or more operating buttons.

According to some embodiments, the user control interface may be functionally associated with the one or more PCBs, allowing control over operation of the endoscope.

According to some embodiments, the operating buttons are magnetically operated.

According to some embodiments, the handle may further include one or more secondary power sources, configured to receive power from a primary external power source and distribute power within the handle.

According to some embodiments, the handle may further include one or more of: a micro-controller (μcontroller), a universal asynchronous receiver-transmitter (UART), a mounting element, a connecting element, or any combination thereof.

According to some embodiments, there is provided an endoscope comprising the handle disclosed herein, and an elongated compatible shaft.

According to some embodiments, the endoscope may include at least one camera and at least one illumination component associated with said at least one camera located at the shaft distal section.

According to some embodiments, the endoscope may include at least two cameras.

According to some embodiments, each of the cameras may be connected to a flexible PCB or a suitable transmitting wire, extending along the shaft of the endoscope and connected to the one or more PCBs located in the handle.

According to some embodiments, the at least two cameras may include a front camera on a distal tip of the shaft and a first side-camera. According to some embodiments, the at least two cameras may further include a second side-camera, wherein the first side-camera and the second side-camera are positioned on opposite sides of the shaft, and wherein the first side-camera is positioned distally relative to the second side-camera.

According to some embodiments, the at least two cameras provide at least about 270 degrees horizontal field-of-view (FOV) of a target area within an anatomical cavity into which the elongated shaft is inserted.

According to some embodiments, the at least one illumination component is or comprises a discrete light source.

According to some embodiments, the shaft may be rigid or semi rigid.

According to some embodiments, there is provided a method for maintaining signal quality, preventing degradation of a signal and/or limiting degradation information conveyed by one or more parallel signals output by one or more sensors of an endoscope, the method comprising converting each of the one or more parallel signals to one or more serial signals in a handle of the endoscope.

According to some embodiments, the method may further include conveying or relaying said one or more serial signals generated in the handle to a main control unit functionally associated with the handle.

According to some embodiments, there is provided a method of converting a parallel signal output from one or more sensors of an endoscope, to a serial signal, and relaying said serial signal to a remote location, wherein the conversion is performed in a handle of the endoscope.

According to some embodiments, the parallel signal may be obtained at a distal end of the endoscope and converted to a serial signal in the handle.

According to some embodiments, the serial signal generated in the handle of the endoscope is relayed to an external control unit, functionally associated with the distal end of the handle.

According to some embodiments, the one or more parallel signals received at the converter may be at 8-12 bit, at 9-11 bit, at 10 bit.

According to some embodiments, the one or more corresponding serial signals may be transmitted at a rate of about 1-2 Giga bits per second, or a rate of about 1.5 Giga bits per second.

According to some embodiments, there is provided a handle of a multi-camera endoscope, the handle includes a user interface activating unit configured to control one or more operating parameters of the endoscope, the user interface activating unit, also referred to as an activation unit includes external operation/control buttons, configured to interact with a corresponding internal activation element, said activation having corresponding sensors configured to interact with the external operation buttons to convey operating commands to one or more cameras of the endoscope, to thereby control one or more operation parameters of the endoscope.

According to some embodiments, there is provided a handle of a multi-camera endoscope, the handle includes a user interface activating unit configured to control one or more operating parameters of the endoscope, the user interface activating unit, also referred to as an activation unit, comprises one or more external control buttons, and an internal activation element, said internal activation element includes one or more sensors, each of said sensors is configured to interact with a corresponding external control button, to convey operating commands (instructions) to the endoscope, to thereby facilitate control of one or more operation parameters of the endoscope.

According to some embodiments, the one or more external control buttons may include a magnetic region.

According to some embodiments, the one or more internal activation element sensors comprise magnetic sensors. According to some embodiments, the magnetic sensors are selected from analog magnetic sensor and linear magnetic sensor.

According to some embodiments, the magnetic sensor may be an analog magnetic sensor, located at a distance of about 0.5 to about 0.7 millimeters from the magnetic region of the corresponding external control button at a non-activated state (off state).

According to some embodiments, the analog magnetic sensor may be activated when the distance between the analog sensor and the magnetic region of the corresponding external control button is changed by about 0.6 millimeters

According to some embodiments, the linear magnetic sensor may be activated when a change is voltage activation of the linear magnetic sensor is sensed. According to some embodiments, the change in voltage may be in the range of about 0.2-0.5V. According to some embodiments, the change in voltage may be about 0.3V.

According to some embodiments, the external control button and the corresponding internal activating element do not physically interact (i.e., they do not touch).

According to some embodiments, the handle may include at least two external control buttons and at least two corresponding internal activating buttons.

According to some embodiments, the handle may include at least three external control buttons and at least three corresponding internal activating buttons.

According to some embodiments, the internal activation element may be mounted on a Printed Circuit Board (PCB), located within the handle.

According to some embodiments, the PCB may be a rigid PCB, a flexible PCB, or both. According to some embodiments, the PCB may be placed in parallel to a longitudinal axis of the handle.

According to some embodiments, the distance (vertical distance, height) of the internal activating element from the internal upper surface of the handle is determined to allow optimized magnetic field between the magnetic region of the external control buttons and the corresponding magnetic sensor of the internal activation element.

According to some embodiments, the handle may include more than one PCB.

According to some embodiments, may include at least two PCBs

According to some embodiments, the handle is functionally associated with an external control unit, said external control unit is connected to a proximal end of the handle.

According to some embodiments, the endoscope may include one or more optical sensors located at a distal section of a shaft of the endoscope and wherein each of the one or more optical sensors is associated with a lens assembly to obtain a camera, the shaft being mounted on the distal end of the handle.

According to some embodiments, the endoscope may include one or more illumination component located at the distal section of the shaft of the endoscope.

According to some embodiments, the handle may further include one or more secondary power sources, configured to receive power from a primary external power source and distribute power within the handle and/or from the handle to the distal section of the shaft of the endoscope.

According to some embodiments, the handle may further include one or more of: a micro-controller (μcontroller), a universal asynchronous receiver-transmitter (UART), a mounting element, a connecting element, or any combination thereof.

According to some embodiments, the control of the operating parameters may include controlling the operation of the one or more optical sensors, one or more lens assemblies, cameras, illumination component, or any combination thereof.

According to some embodiments, the operating parameters may include: zoom-in, zoom out, image-capture, freezing an image, recording an image or a video, saving an image of a videos, changing the intensity of the illumination component, manipulating between video streams of the multi-camera endoscope to be displayed on a monitor, or any combination thereof.

According to some embodiments, the handle is essentially sealed, such that external fluids are prevented from entering internal space of the handle.

According to some embodiments, there is provided an endoscope which includes the handle as disclosed herein, and an elongated compatible shaft.

According to some embodiments, the endoscope may include at least one camera and at least one illumination component associated with said at least one camera located at the shaft distal section. According to some embodiments, the endoscope may include at least two cameras. According to some embodiments, each of the cameras may be connected to a flexible PCB or a suitable transmitting wire, extending along the shaft of the endoscope and connected to the one or more PCBs located in the handle.

According to some embodiments, the at least two cameras of the endoscope includes a front camera on a distal tip of the shaft and a first side-camera. According to some embodiments, the at least two cameras may further include a second side-camera, wherein the first side-camera and the second side-camera are positioned on opposite sides of the shaft, and wherein the first side-camera is positioned closely relative to the second side-camera.

According to some embodiments, the least two cameras may provide at least about 270 degrees horizontal field-of-view (FOV) of a target area within an anatomical cavity into which the elongated shaft is inserted.

According to some embodiments, the at least one illumination component is or includes a discrete light source.

According to some embodiments, the shaft of the endoscope may be rigid or semi rigid.

According to some embodiments, there is provided a method for controlling operation of a medical imaging system including a multi-camera endoscope, the method comprising conveying operation instructions from user interface activating unit, located within a handle of the multi-camera endoscope, wherein the operation instructions are further conveyed to a main control unit, and/or to one or more cameras and/or to one or more illumination components located at a distal end of the endoscope. According to some embodiments, the operating parameters may include one or more of: zoom in, zoom out, image capture, freezing an image, saving an image, recording a video, changing the intensity of the light source, manipulating between video streams of the multi-camera endoscope to be displayed on a monitor, or any combination thereof.

According to some embodiments, there is provided a method for controlling operation of an endoscope, the method includes conveying operation instructions from one or more external control buttons located on an external surface of a handle of an endoscope to one or more corresponding sensors of an internal activation element, located within the handle, wherein the external control button and the corresponding activation element sensor to do not physically interact, wherein the operation instructions are further conveyed to one or more cameras and/or one or more illumination components, located at a distal end of a shaft of the endoscope.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus. In some embodiments, a computer may include of the apparatuses may include FPGA, microcontrollers, DSP and video ICS.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 99% and 101% of the given value. In such embodiments, for example, the statement “the length of the element is equal to about 1 millimeter” is equivalent to the statement “the length of the element is between 0.99 millimeters and 1.01 millimeters”.

As used herein, according to some embodiments, the terms “substantially” and “about” may be interchangeable.

It should also be noted that a plurality of terms, as follows, appearing in this specification are used interchangeably to apply or refer to similar components and should in no way be construed as limiting: A “user interface activating unit” may also be referred to as “activation unit” and or “user control interface”.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1.-50. (canceled)

51. A handle of a multi-camera endoscope, the handle comprising a user interface activating unit configured to control one or more operating parameters of the endoscope, the user interface activating unit comprises one or more external control buttons comprising a magnetic region, and an internal activating element, said internal activating element comprises one or more magnetic sensors, each of said one or more magnetic sensors is configured to interact with a corresponding external control button, to convey operating commands to the endoscope, to thereby facilitate control of one or more operation parameters of the endoscope.

52. The handle according to claim 51, wherein the one or more magnetic sensors are selected from an analog magnetic sensor and a linear magnetic sensor; wherein the analog magnetic sensor is located at a distance of about 0.5 to about 0.7 millimeters from the magnetic region of the corresponding external activating/control button at a non-activated state (off state); and/or
wherein the analog magnetic sensor is activated when the distance between the analog sensor and the magnetic region of the corresponding external control button is changed by about 0.6 millimeters

53. The handle according to claim 52, wherein the linear magnetic sensor is activated when a change is voltage activation of the linear magnetic sensor is sensed, wherein the change in voltage is in the range of about 0.2-0.5V.

54. The handle according to claim 51, wherein the external control button and the corresponding internal activating element do not physically interact.

55. The handle according to claim 51, comprising at least two external control buttons and at least two corresponding internal activating elements.

56. The handle according to claim 51, wherein the internal activating element is mounted on a Printed Circuit Board (PCB), located within the handle.

57. The handle according to claim 51, further comprising a converter configured to receive one or more parallel signals and transform said one or more parallel signals to one or more corresponding serial signals, wherein the parallel signals are output by one or more optical sensors of the endoscope, said converter is further configured to transmit or relay the serial signals to an external control unit, wherein the converter is configured to relay the serial signals to a utility cable connected to the external control unit that is functionally associated with the handle.

58. The handle according to claim 57, wherein the one or more parallel signals received at the converter are at 8-12 bit, at 9-11 bit, and/or at 10 bit; and/or wherein the one or more corresponding serial signals may be transmitted at a rate of about 1-2 Giga bits per second, or a rate of about 1.5 Giga bits per second.

59. The handle according to claim 57, wherein the one or more optical sensors are located at a distal section of an elongated shaft of the endoscope and wherein each of the one or more optical sensors is associated with a lens assembly located at distal section to obtain a camera, the elongated shaft being mounted on the distal end of the handle.

60. The handle according to claim 57, wherein the transmitting to the control unit is direct or via one or more intermediate elements and/or wherein the converter comprises Field Programmable Gate Array (FPGA).

61. The handle according to claim 51, wherein the handle comprises at least two PCBs, said PCBs comprising rigid PCB, flexible PCB, or both; wherein the at least two PCB's are placed parallel along a longitudinal axis of the handle.

62. The handle according to claim 57, wherein each of the one or more optical sensors is connected to transferring means, extending along the elongated shaft of the endoscope and connected to the converter, wherein the transferring means comprise cables, flexible PCBs, rigid PCBs, flex-rigid PCBs or any combination therefore.

63. The handle according to claim 51, wherein the handle further comprises one or more secondary power sources, configured to receive power from a primary external power source and distribute power within the handle and/or wherein the handle further comprises one or more of: a micro-controller (μcontroller), a universal asynchronous receiver-transmitter (UART), a mounting element, a connecting element, or any combination thereof.

64. The handle according to claim 51, wherein the handle is functionally associated with an external control unit, said external control unit is connected to a proximal end of the handle.

65. The handle according to claim 51, wherein the control of the operating parameters comprises controlling the operation of the one or more optical sensors, one or more lens assemblies, cameras, illumination component, or any combination thereof, wherein the operating parameters comprises: zoom-in, zoom out, image-capture, freezing an image, recording an image or a video, saving an image of a videos, changing the intensity of the illumination component, manipulating between video streams of the multi-camera endoscope to be displayed on a monitor, or any combination thereof.

66. The handle according to claim 51, wherein the handle is essentially sealed, such that external fluids are prevented from entering internal space of the handle; optionally wherein the handle further comprises a leak tester.

67. An endoscope comprising the handle according to claim 51, and an elongated compatible shaft, said endoscope comprises a front camera on a distal tip of the elongated shaft, a first side-camera and a second side-camera, wherein the first side-camera and the second side-camera are positioned on opposite sides of the elongated shaft, and wherein the first side-camera is positioned closely relative to the second side-camera; wherein the provide at least about 270 degrees horizontal field-of-view (FOV) of a target area within an anatomical cavity into which the elongated shaft is inserted; and at least one illumination component.

68. A method for controlling operation of a multi camera endoscope, the method comprising conveying operation instructions from one or more external activating/control buttons located on an external surface of a handle of the multi camera endoscope to one or more corresponding sensors of an internal activating element, located within the handle, wherein the external control button and the corresponding activating element to do not physically interact, wherein the operation instructions are further conveyed to one or more cameras and/or one or more illumination components, located at a distal end of an elongated shaft of the endoscope.

69. The method according to claim 68, wherein the one or more external control buttons comprise a magnetic region, wherein the one or more sensors comprise magnetic sensors, selected from analog magnetic sensor and linear magnetic sensor.

70. The method according to claim 68, wherein the operating parameters comprise one or more of: zoom in, zoom out, image capture, freezing an image, saving an image, recording a video, changing the intensity of the light source, manipulating between video streams of the multi-camera endoscope to be displayed on a monitor, or any combination thereof.
